(19) United States
(12) Patent Application Publication
(10) Pub. No.: US 2010/0068824 A1
(43) Pub. Date: Mar. 18, 2010

(54) SENSING METHOD, SENSING DEVICE, INSPECTION CHIP, AND INSPECTION KIT

(75) Inventor: Toshihito KIMURA, Ashigara-kami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Tokyo (JP)

(21) Appl. No.: 12/560,929

(22) Filed: Sep. 16, 2009

(30) Foreign Application Priority Data
Sep. 16, 2008 (JP) 2008-237060

(51) Int. Cl. G01N 33/53; G01N 33/00; B01L 3/00; G06F 19/00

(52) U.S. Cl. 436/501; 422/68.1; 422/61; 702/19; 250/458.1; 356/301

(57) ABSTRACT

A sensing method comprises the steps of: allowing a liquid sample containing an analyte to flow through a channel, applying a force oriented in a given direction normal to a direction in which the liquid sample flows in the channel upon the analyte in a given position of the channel to move the analyte in the given direction so that the analyte is concentrated, causing the liquid sample to flow to a sensing surface forming a part of a wall surface of the channel located downstream of the given position and in the given direction against the channel, the sensing surface securing thereon a binding substance specifically reacting with the analyte, to allow the concentrated analyte to bind to the binding substance, and detecting a quantity of the analyte bound to the binding substance.

The entire contents of the documents cited in this specification are incorporated herein by reference.

### BACKGROUND OF THE INVENTION

The invention relates to a sensing method of detecting an analyte in a liquid sample and measuring the quantity and the concentration thereof, a sensing device for implementing the sensing method, and an inspection chip and an inspection kit used therefor.

Conventionally, immunoassay using a specific reaction between antigen and antibody (antigen-antibody reaction) has been employed as one of the methods of quantifying an analyte in a liquid sample.

Immunoassay is a method whereby an antigen or an antibody is labeled by a labeling substance, the labeled antigen or antibody is used to cause an analyte, which is an antigen or an antibody contained in a sample, to undergo an antigen-antibody reaction, whereupon a complex of the analyte, the antibody, and the labeling substance resulting from that reaction is quantitatively detected.

Such immunoassay has a drawback: when inducing an antigen-antibody reaction by bringing a liquid sample containing an analyte such as an antigen, as exemplified by an antigen-label complex, to an antibody secured to a sensing surface, only the antigen-label complex located closer to the sensing surface (interface) can be bound to the secured antibody because of a low diffusion speed of the antigen-label complex in the liquid sample, thus taking a long time to achieve a sufficient reaction between the analyte and the secured antibody.

Accordingly, when a quantitative measurement should be made in a short period of time, the quantitative measurement could be terminated before the antigen (analyte) sufficiently binds to the secured antibody, making it difficult to achieve a quick, high-sensitivity, and high-accuracy quantitative detection of an analyte in a liquid sample.

Another method and device have been proposed whereby an electric field or a magnetic field is used to concentrate a sample and draw an analyte in the sample to a place where measurement is made.

JP 02-297053 A, for example, describes a method and a device using a sample liquid comprising electrodes, one of which is formed of a sensitive substance in the form of a thin monomolecular layer secured to an electrically conductive surface, whereby an alternating current voltage is applied to the liquid sample so as to act upon polarized components in the sample and draw the specimen to the sensitive substance.

JP 09-304339 A describes a method and a device whereby an electric voltage is applied through a first transparent electrode and a second electrode to an electrophoresis medium containing an analyte labeled by a fluorescent body to electrophorese the analyte in the sample to its interface with the first electrode, whereupon excitation light is applied to the interface to detect the fluorescence generated by the fluorescent body in the analyte as it is excited by the evanescent waves leaking from the interface.

JP 2005-077338 A describes a method and a device whereby a second reactant complex comprising a first reactant-analyte-optical acting component (label) in the liquid sample is localized in a region by using magnetism, whereupon excitation light is applied to a given region containing the localized region to detect the fluorescence excited by leaked evanescent waves and emitted by optical acting components.

JP 2003-527601 A describes a method and a device whereby an analyte or a polar analyte is moved in parallel along a parallel movement passage to which an alternating current field crossing the passage is applied to concentrate the polar analyte in regions where the parallel movement passage and the alternating current field cross.

However, because the electrodes are located just above the measuring region (sensing surface) in the methods and the devices described in JP 02-297053 A and JP 09-304339 A, this configuration requires expensive transparent electrodes not to hinder detection of the fluorescence generated from the fluorescent body for fluorescence measuring or the like. Further, when a transparent electrode is used, surface plasmon is not produced and hence it was difficult to implement the surface plasmon fluorescence measuring method or SPF (surface plasmon enhanced fluorescence) method, which is a fluorescence detection method yielding a yet higher signal-to-noise ratio.

In addition, JP 2005-077338 A has a problem that the antibody is liable to form aggregates depending upon the kind of a labeled antibody selected. Once the antibody forms aggregates on the sensing surface, the antigen detaches, making removal of aggregates impossible. Further, although one piece of antigen should be quantitatively measured with one piece of antibody, detachment of the antigen causes disparity in number between antigen and antibody, making accurate quantitative measurement impossible.

According to the device described in JP 2003-527601 A, although the analyte is concentrated in the regions where the parallel movement passage and the alternating current field cross, the analyte is concentrated not on the sensing interface but along the parallel movement passage. Accordingly, the analyte cannot bind to the antibody secured to the sensing interface in a position distanced from the sensing interface, making accurate quantitative measurement difficult.

Thus, the conventional concentration technique whereby concentration is performed at the sensing surface had problems in terms of costs and the accuracy of quantitative measurements attained. Further, concentration increased non-specific adsorptions of labeled antibody and labeled antigen, which in turn led to increased background noise due to the non-specific adsorptions and, hence, to decreased signal-to-noise ratio, resulting in low-sensitivity.

### SUMMARY OF THE INVENTION

Thus, an object of the present invention is to overcome the above problems associated with the prior art and provide a sensing method and a sensing device enabling performing a high-sensitivity quantitative measurement at low costs.

Another object of the invention is to provide an inspection chip and an inspection kit used for such a sensing method and sensing device.

A sensing method according to the present invention comprises the steps of: allowing a liquid sample containing an analyte to flow through a channel, applying a force oriented in a given direction normal to a direction in which the liquid sample flows in the channel upon the analyte in a given position of the channel to move the analyte in the given direction so that the analyte is concentrated, causing the liquid sample to flow to a sensing surface forming a part of a wall surface of the channel located downstream of the given position and in the given direction against the channel, the sensing surface securing thereon a binding substance specifically reacting with the analyte, to allow the concentrated analyte to bind to the binding substance, and detecting a quantity of the analyte bound to the binding substance.

A sensing device according to the present invention comprises: a channel for allowing a liquid sample containing an analyte to flow therethrough, concentration means for applying a force oriented in a given direction normal to a direction in which the liquid sample flows upon the analyte in a given position of the channel to move the analyte in the given direction so that the analyte is concentrated, a sensing surface forming a part of a wall surface of the channel located downstream of the given position and in the given direction against the channel, the sensing surface securing thereon a binding substance specifically reacting with the analyte, and detection means for detecting a quantity of the analyte bound to the binding substance.

An inspection chip according to the present invention comprises: a channel substrate including a channel through which a liquid sample containing an analyte is allowed to flow and formed with a feed inlet for feeding the liquid sample to the channel and a discharge outlet for discharging the liquid sample from the channel, a sensing surface forming a part of a bottom surface of the channel between the feed inlet and the discharge outlet of the channel substrate, a binding substance secured to the sensing surface and specifically reacting with the analyte, and a concentration region located in the channel on a side of the sensing surface closer to the feed inlet and provided to apply a force to the analyte to move the analyte toward the bottom surface of the channel for concentration.

An inspection kit according to the present invention comprises: such an inspection chip and an ampoule containing a labeling solution including a labeling substance for detection for labeling the analyte for detection.

### BRIEF DESCRIPTION OF THE DRAWINGS

FIG. 1 is a block diagram illustrating a configuration of a sensing device according to Embodiment 1 of the invention.

FIG. 2 is a top plan view illustrating a light source, incidence optics, and an inspection chip according to Embodiment 1.

FIG. 3 is a cross section taken along line A-A in FIG. 2.

FIG. 4 is a cross section taken along line B-B in FIG. 2.

FIG. 5 is a top plan view illustrating the inspection chip to which a suction unit is connected.

FIGS. 6A to 6E are top plan views of the inspection chip illustrating progressive stages of a method of detecting an analyte using a sandwich technique.

FIGS. 7A to 7G are side cross sections of the inspection chip illustrating progressive stages of a method of detecting an analyte using a sandwich technique.

FIG. 8 is a view schematically illustrating a configuration of a fluorescent substance.

FIGS. 9A to 9C are views for explaining the principle of competition technique.

FIGS. 10A to 10G are side cross sections of an inspection chip illustrating progressive stages of a method of detecting an analyte using the competition technique.

FIG. 11 is a view illustrating a part of a configuration of a sensing device according to Embodiment 2 of the invention.

FIGS. 12A to 12C illustrate specific examples of microstructures used in Embodiment 2.

FIG. 13 is a view illustrating a part of a configuration of a sensing device according to a variation of Embodiment 2 of the invention.

FIG. 14 is a view illustrating a part of a configuration of a sensing device according to another variation of Embodiment 2 of the invention.

FIG. 15 is a view illustrating a part of a configuration of a sensing device according to Embodiment 3 of the invention.

FIG. 16 is a view illustrating a configuration of an inspection kit according to Embodiment 4 of the invention.

FIGS. 17A to 17K are side cross sections of an inspection chip illustrating progressive stages of a method of detecting an analyte using the inspection kit according to Embodiment 4.

### DETAILED DESCRIPTION OF THE INVENTION

The sensing method, the sensing device, the inspection chip and the inspection kit of the invention are described in detail below based on the preferred embodiments illustrated in the accompanying drawings.

### Embodiment 1

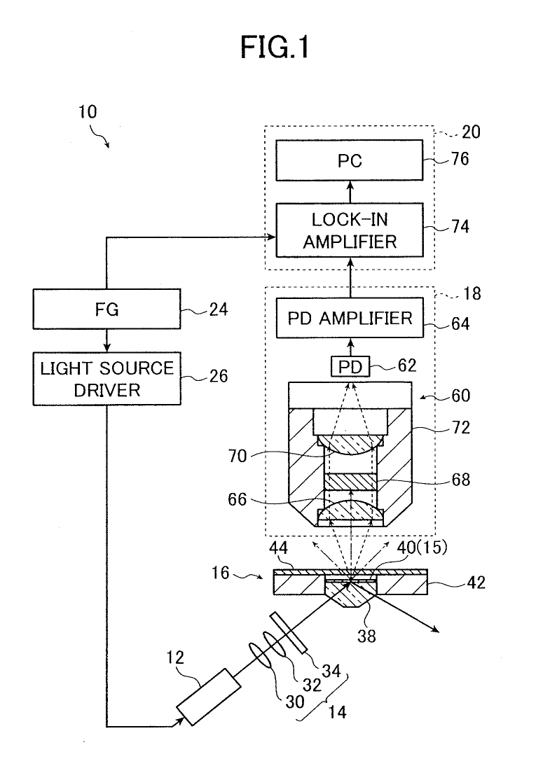

FIG. 1 illustrates a schematic configuration of a sensing device 10 according to Embodiment 1 of the invention.

Basically, the sensing device 10 implements a substance detection method for detecting the binding quantity of an analyte in a liquid sample using antigen-antibody reaction in order to measure the quantity and concentration of the analyte.

The sensing device 10 comprises a light source 12 for emitting light having a given wavelength, incidence optics 14 for guiding and condensing the light emitted from the light source 12 (also referred to as “excitation light” below), and an inspection chip 16 for holding a liquid sample containing an analyte (antigen). The inspection chip 16 comprises a sensing surface 15 that is hit by the light condensed by the incidence optics 14. The sensing device 10 further comprises a light detection unit 18 for detecting the light emitted from the sensing surface 15 of the inspection chip 16, a computation unit 20 for digitizing the detection signal from the light detection unit 18 to determine the presence/absence of the analyte, the quantity and the concentration thereof, a concentration unit described later herein for concentrating the liquid sample containing an analyte, and a suction unit described later for supplying a concentrated liquid sample to the sensing surface 15 of the inspection chip 16.

The sensing apparatus 10 further comprises a function generator (also referred to as “FG” below) 24 for generating a modulation signal for modulating the intensity of the excitation light emitted from the light source 12 and a light source driver 26 for driving the light source 12 according to the modulation signal generated by the FG 24.

The modulation signal generated from the FG 24 is a repetitive pulse signal having high level and low level voltages. The FG 24 supplies the modulation signal to the light source driver 26, which causes an electric current based on the modulation signal to flow to the light source 12, which in turn generates excitation light modulated according to the modulation signal. The modulation signal supplied from the FG 24 to the light source 26 is also supplied to the computation unit 20. The computation unit 20 extracts only the signal that is in synchronism with the modulation signal from the output of the light detection unit 18 to make a judgment on the analyte.

Although not shown, the inspection chip 16 and the other components of the sensing apparatus 10 are supported by a support structure to fix their relative positions.

The light source 12 is a light emission device that emits light having a given wavelength. The light source 12 may for example be a semiconductor laser, an LED, a lamp, or an SLD.

The incidence optics 14 comprises a collimator lens 30, a cylindrical lens 32, and a polarizing filter 34, which are disposed in that order on the optical path of the excitation light, the collimator lens 30 being the closest to the light source 12. Therefore, the light emitted from the light source 12 passes through the collimator lens 30, the cylindrical lens 32, and the polarizing filter 34 in that order and then enters the inspection chip 16.

The collimator lens 30 collimates the light emitted from the light source 12 and diffusing radially at a given angle.

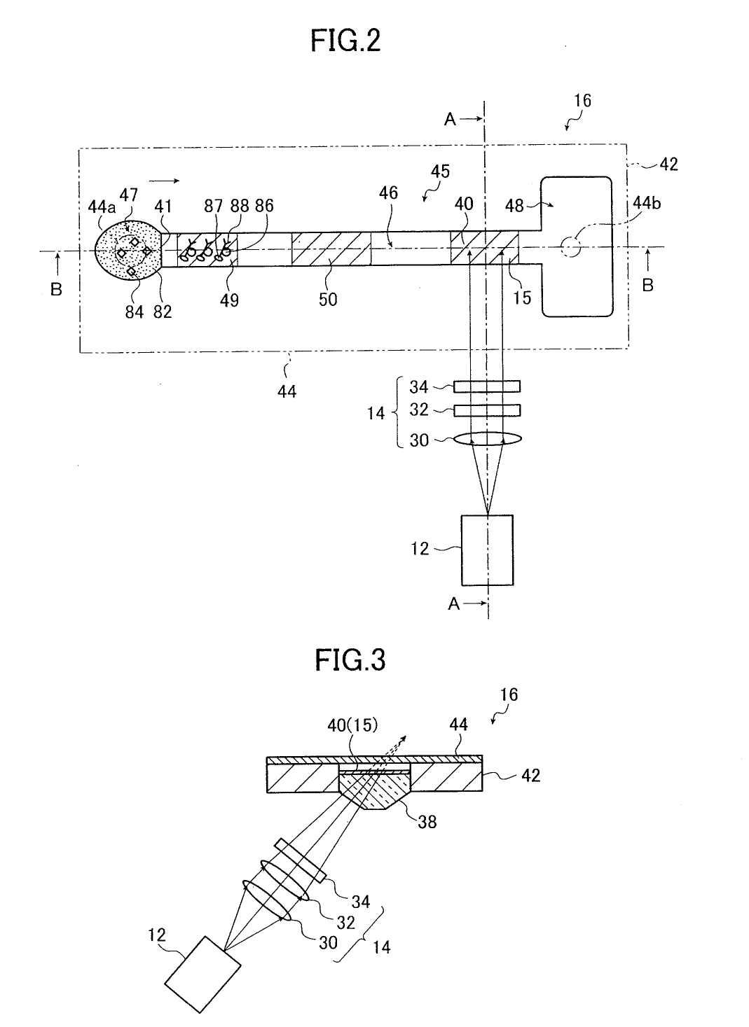

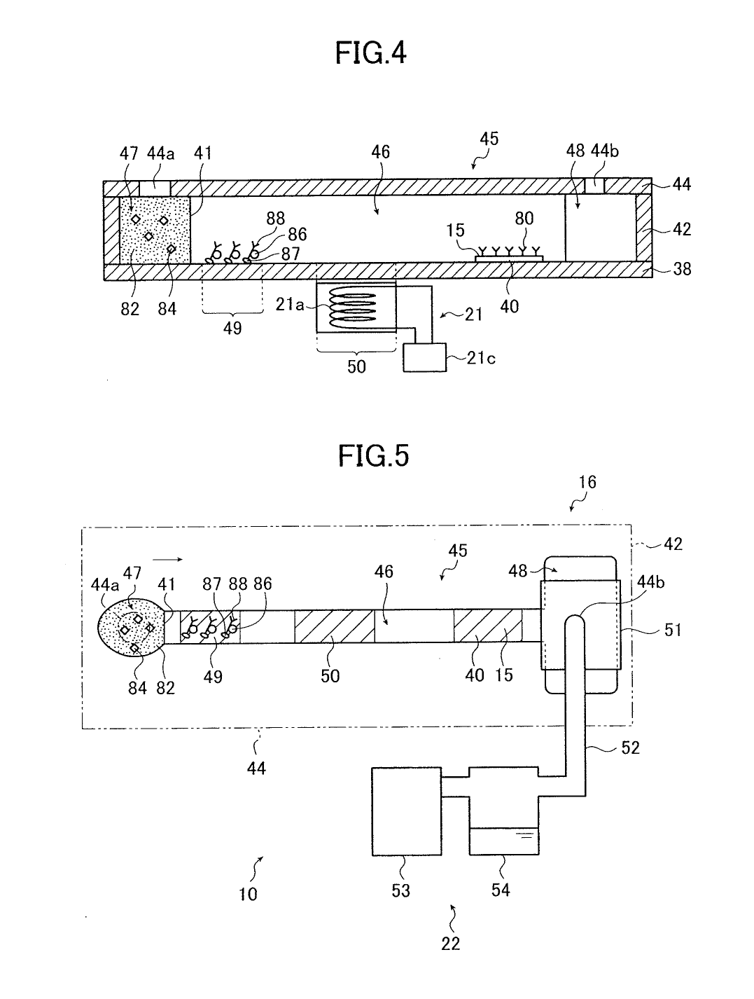

As illustrated in FIGS. 2 and 3, the cylindrical lens 32 is a columnar lens whose axis extends parallel to the length of a channel of the inspection chip 16 described later. The cylindrical lens 32 condenses the light collimated by the collimator lens 30 only in the plane normal to the axis of the column (plane parallel to the plane represented in FIG. 3).

The polarizing filter 34 p-polarizes the light passing through it with respect to a reflection surface of the inspection chip 16 described later.

The inspection chip 16 comprises a prism 38, a metal film 40 having a surface forming the sensing surface 15, a channel substrate 42, and a transparent cover 44 (also referred to simply as “cover” below). A liquid sample containing an analyte is placed on the surface of the metal film 40 that in turn is formed on one plane of the prism 38, i.e., the sensing surface 15.

The prism 38 is in the form of a generally triangular prism having a cross section shaped like an isosceles triangle (to be more exact, the prism is in the form of a hexagonal cylinder as obtained by cutting off the apices of the isosceles triangle in planes normal and parallel to the base of the isosceles triangle); this prism is disposed on the optical path of the light that is emitted from the light source 12 and condensed by the incidence optics 14.

The prism 38 is disposed in a position and orientation such that, as seen in its cross section, the light condensed by the incidence optics 14 enters through the plane that is defined by one of the two oblique sides of the isosceles triangle, is then reflected by the plane that is defined by the base of the isosceles triangle, and emitted through the plane that is defined by the other of the two oblique sides of the isosceles triangle.

While the prism 38 may be formed of a known transparent resin or optical glass that is a dielectric, a resin is preferred to optical glass in terms of costs.

Examples of resins that may be used to form the prism 38 include polymethyl methacrylates (PMMA), polycarbonates (PC), amorphous polyolefins (APO) containing cycloolefin, and ZEONEX (trademark) 330R (refraction index 1.50), a product of ZEON CORPORATION.

Materials that may be used as the optical glass include BK7 and quart.

The metal film 40 is a thin metal film that is formed on at least a part of that surface of the prism 38 that is defined by the base of the isosceles triangle (specifically, the part is an area that includes a region illuminated by the light that enters the prism 38).

The metal film 40 may be formed of a metal such as Au, Ag, Cu, Pt, Ni and Al. In order to suppress its reaction with the liquid sample, Au or Pt is preferably used.

The metal film 40 may be formed by a variety of methods; for example, it may be formed on the prism 38 by sputtering, evaporation, plating, or pasting.

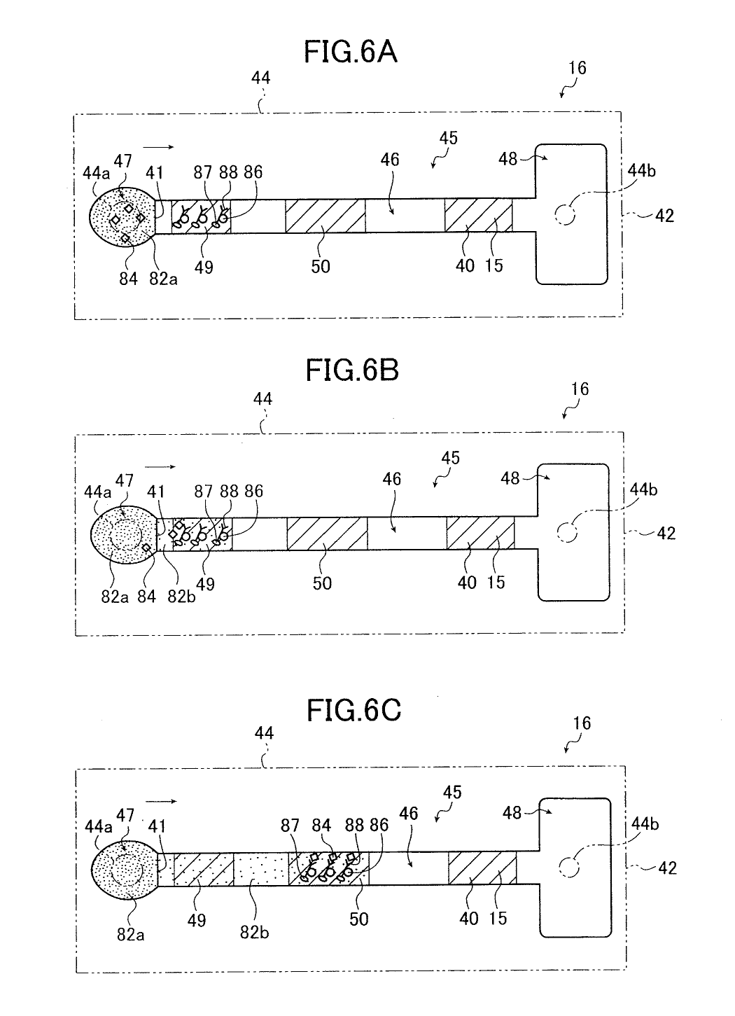

As illustrated in FIG. 4, the metal film 40 has a primary antibody 80 secured to its surface to provide the sensing surface 15. The primary antibody 80 is a specific binding substance that specifically binds to an analyte 84.

The channel substrate 42 is a plate member disposed on the plane defined by the base of the isosceles triangle of the prism 38 and has a channel 45 formed to provide a passage for feeding a liquid sample 82 to the metal film 40 as illustrated in FIG. 2.

The metal film 40 is exposed on a part of the bottom surface of the channel 45 to provide the sensing surface 15. The channel 45 consists of a linear portion 46 formed across the metal film 40, a leading end portion 47 that is formed at one end of the linear portion 46 to serve as a liquid reservoir into which the liquid sample 82 is fed for measurement, and a terminal end portion 48 that is formed at the other end of the linear portion 46 to serve as a liquid reservoir that is reached by the liquid sample 82 that has passed through the linear portion 46. At the boundary between the leading end 47 and the linear portion 46 of the channel 45 is provided a filter 41 that allows the passage of the analyte 84 such as an antigen and the solvent in the liquid sample 82, for example, but does not allow the passage of particles larger than the analyte 84.

The linear portion 46 also comprises a secondary antibody deposit region 49 and a concentration region 50 on the upstream side of the metal film 40, i.e., closer to the leading end 47, the former being located upstream of the latter. The secondary antibody deposit region 49 is a region where the secondary antibody 88 labeled by a fluorescent substance 86 and magnetic particles 87 is placed. The concentration region 50 is provided for concentration. The secondary antibody 88 is a specific binding substance that specifically binds to the analyte 84.

A transparent cover 44 is a transparent plate member connected to the plane of the channel substrate 42 opposite from the prism 38 and closes the channel 45 formed in the channel substrate 42.

The transparent cover 44 has a feed inlet 44a and an air outlet (discharge outlet) 44b in positions corresponding to the leading end portion 47 of the channel 45 and the terminal end 48 of the channel 45. The feed inlet 44a is an aperture for feeding the liquid sample 82 to the channel 45; the air outlet 44b is an aperture for discharging air to cause the liquid sample 82 in the channel 45 to flow downstream. The feed inlet 44a and the air outlet 44b may be provided with lids that can be opened and closed.

While the prism 38 provided with the metal film 40 and the channel substrate 42 are preferably formed in one piece, the invention is not limited this way. The invention permits a configuration that the channel substrate 42 is provided with a bottom portion so as to form a bottom surface of the inspection chip 16 such that the entire bottom portion or at least a part thereof where the metal film 40 forming the sensing surface 15 is located is formed of a transparent dielectric film, and the top surface of the prism 38 where the metal film 40 is disposed is allowed to be in contact with the underside of the transparent dielectric film, thus providing the prism 38 and the inspection chip 16 discretely.

The light source 12, the incidence optics 14, and the inspection chip 16 are arranged in such relative positions that the light passing through the incidence optics 14 to enter the prism 38 through one of its panes is totally reflected by the metal film 40 and allowed to leave through the other plane of the prism 38.

Thus, when the excitation light is caused to enter the prism 38 by the light source 12 and the incidence optics 14 in such a manner that the excitation light is totally reflected by the metal film 40 of the inspection chip 16, evanescent light (also referred to as evanescent waves below) exudes on the surface of the metal film 40 on the side of the metal film 40 facing the channel 45 (the surface opposite from the prism 38), i.e., the sensing surface 15. These evanescent waves excite surface plasmons in the metal film 40. The surface plasmons in turn produce an electric field distribution on the surface of the metal film 40, forming enhanced field regions. When the fluorescent substance 86 is present in the area where the evanescent waves exude, the fluorescent substance 86 is excited by the evanescent waves to generate fluorescence.

The fluorescence is reinforced by the effect of field enhancement by the surface plasmons that are present in areas substantially the same areas as where the evanescent waves have exuded.

Note that the fluorescent substance 86 that is located outside an area where the evanescent waves have exuded is not excited and hence does not generate fluorescence.

The illumination method whereby fluorescence is generated by the intermediate of evanescent waves is called the evanescent excitation illumination method.

The light detection unit 18 comprises a detection optics 60, a photodiode (hereinafter referred to as PD) 62, and a photodiode amplifier (hereinafter referred to as PD amplifier) 64, and it detects light occurring from the sensing surface 15 of the metal film 40 in the inspection chip 16 as the metal film 40 of the inspection chip is irradiated with the excitation light emitted from the light source 12.

The detection optics 60 comprises a first lens 66, a cut-off filter 68, a second lens 70, and a support member 72 supporting these components and condenses the light emitted from the sensing surface 15 so that the light enters the PD62. In the detection optics 60, the first lens 66, the cut-off filter 68, and the second lens 70 are disposed in this order with a given distance provided between them, the first lens 66 being the closest to the metal film 40.

The first lens 66 is a collimator lens provided opposite the metal film 40; it collimates the light that occurs on the metal film 40 and reaches the first lens 66.

The cut-off filter 68 has such a characteristic of selectively cutting off a light component that has the same wavelength as the excitation light emitted from the light source 12 but transmitting a light component having a different wavelength than the excitation light (e.g., fluorescence originating from the fluorescent substance 86). Thus, the cut-off filter 68 transmits only that portion of the collimated light from the first lens 66 that has a different wavelength than the excitation light.

The second lens 70 is a condenser lens that condenses the light passing through the cut-off filter 68 and allows it to enter the PD 62.

The support member 72 is a holder member that integrally holds the first lens 66, the cut-off filter 68 and the second lens 70 spaced a given distance from each other.

The PD 62 is an optical detector that converts received light into an electric signal; it converts the incoming light that has been condensed by the second lens 70 into an electric signal. The PD 62 sends the electric signal to the PD amplifier 64 as a detection signal.

The PD amplifier 64 is an amplifier that amplifies the detection signal; it amplifies the detection signal sent from the PD 62 and sends the amplified detection signal to the computation unit 20.

The computation unit 20 comprises a lock-in amplifier 74 and a PC (computation section) 76 and calculates the mass of the analyte, its concentration and the like from the detection signal.

The lock-in amplifier 74 is supplied with a modulation signal generated by the FG 24 as reference signal. The lock-in amplifier 74 amplifies that component of the detection signal that has the same frequency as a reference signal; it amplifies that component of the detection signal amplified by the PD amplifier 64 which is synchronous with the reference signal sent from the FG 24. The detection signal amplified by the lock-in amplifier 74 is outputted to the PC 76.

The PC 76 converts the detection signal fed from the lock-in amplifier 74 into a digital signal and detects the concentration of the analyte in the sample according to the signal obtained by the conversion. The concentration of the analyte in the sample can be calculated from the relationship between the number of pieces of the analyte and the liquid volume. The number of pieces of the analyte can be calculated from a calibration curve that is previously worked out on the basis of the relationship between the intensity of the detection signal and the number of pieces of the analyte using a known number of pieces of the analyte. The concentration of the analyte can be computed easily and accurately by keeping constant the volume of the liquid sample fed to the channel 45 of the substrate 42 of the inspection chip 16 (or by designing to ensure that a constant volume be fed).

As illustrated in FIG. 4, a concentration unit 21 is disposed close to the concentration region 50 located upstream of the sensing surface 15. The concentration unit 21 applies a force acting toward and normal to the bottom surface of the channel 45, through which the liquid sample 82 is fed, to withdraw the analyte 84 in the liquid sample 82 toward the bottom surface of the channel 45 (on the side on which the sensing surface 15 is disposed) to achieve concentration. The concentration unit 21 comprises an electromagnetic coil 21a disposed just below the concentration region 50. The electromagnetic coil 21a has an axis normal to the bottom surface of the inspection chip 16. Upon passing a given level of electric current through the electromagnetic coil 21a to generate a magnetic field in the concentration region 50, a magnetic force directed toward the bottom surface of the inspection chip 16 acts upon the magnetic particles 87 labeling the second antibody 88 bound to the analyte 84 to move the analyte 84 toward the bottom surface of the inspection chip 16, thus achieving concentration.

Provided with the concentration unit 21 having the electromagnetic coil 21a, the illustrated example uses the secondary antibody 88 labeled by the fluorescent substance 86, which is a labeling substance for optical detection, and the magnetic particles 87, which are a labeling substance for concentration, to achieve concentration of a complex composed of the analyte 84 and the secondary antibody 88 labeled by the fluorescent substance 86 and the magnetic particles 87. The fluorescent substance 86 may be replaced by fluorescent beads and the magnetic particles 87 may be replaced by a magnetic material such as magnetic beads, or the fluorescent substance 86 and the magnetic particles 87 may be replaced by fluorescent magnetic beads made of polyethylene or the like to achieve concentration of a complex composed of the secondary antibody 88 labeled by fluorescent magnetic beads and the analyte 84 such as an antigen.

The concentration unit is not limited to the above configuration and may use any of a variety of concentration methods and means for concentrating the liquid sample 82, provided that the analyte 84 can be moved toward the bottom surface of the inspection chip 16 for concentration. For example, concentration may be started and stopped by introducing and withdrawing a magnet to and from the concentration unit. Alternatively, an electric field oriented toward the bottom surface of the channel 45 of the inspection chip 16, for example, may be generated as will be described to produce Coulomb's force acting upon the analyte 84 to achieve concentration. In this case, the analyte 84 is preferably labeled by such a labeling substance for concentration that increases Coulomb's force acting upon the analyte 84 by means of electric field instead of the magnetic particles 87.

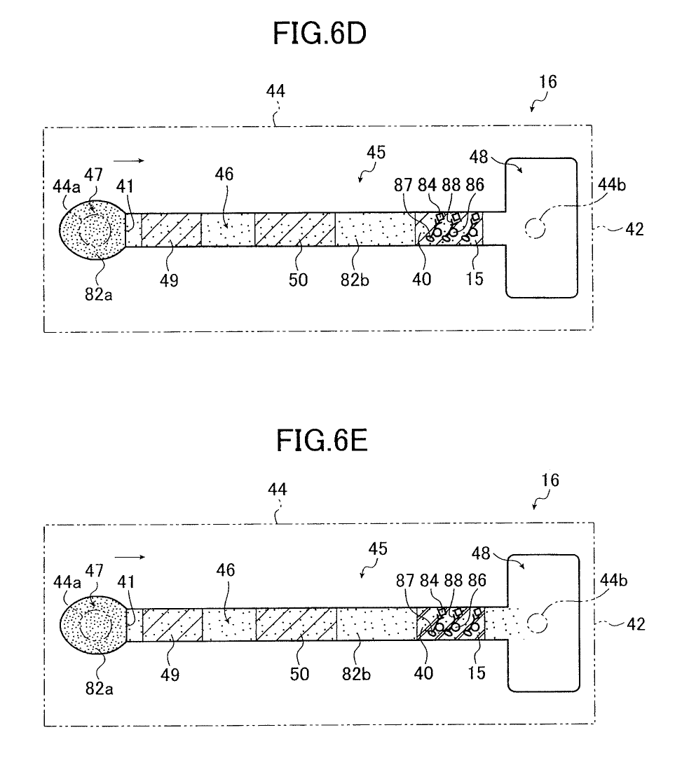

As illustrated in FIG. 5, a suction unit 22 is connected to the inspection chip 16. The suction unit 22 accelerates the reaction for sensing a substance (antigen-antibody reaction) and shortens the detection time. Specifically, the suction unit 22 effectively forces the liquid sample 82 fed from the feed inlet 44a of the cover 44 of the inspection chip 16 and passed through the filter 41 to flow through the channel 45 downstream. The suction unit 22 comprises a connection member 51 connected to the air outlet 44b formed in the cover 44 in a position thereof corresponding to the terminal end 48 of the channel 45 of the inspection chip 16, a tube 52 connected to the connection member 51, a pump 53 connected to the connection member 51 through the tube 52, and a waste liquid tank 54 provided at a part of the tube 52. The suction unit 22 sucks the liquid sample 82 in the channel 45 from the terminal end 48.

The connection member 51 is a plate member disposed on the cover 44 so as to close the air outlet 44b of the cover 44. The connection member 51 may be formed of any of various materials as appropriate. In this embodiment, it is formed of PDMS (polydimethylsiloxane).

The tube 52 is a tubular member having one end thereof connected to the connection member 51 and the other end connected to the pump 53.

The pump 53 is a suction pump that sucks the inside of the terminal end portion 48 through the tube 52 from the air outlet 44b of the cover 44, to which the connection member 51 is connected, to draw the liquid sample 82 in the terminal end portion 48 and the linear portion 45 into the tube 52.

The waste liquid tank 54 is disposed at a part of the tube 52 and stores the liquid sample 82 sucked by the pump 53 from the terminal end portion 48.

The suction unit 22 is not limited to the above configuration and one may use any of various suction means capable of sucking the liquid sample; it may for example be a syringe pump.

Now, the effects and operations of the illustrated sensing device 10 will be described.

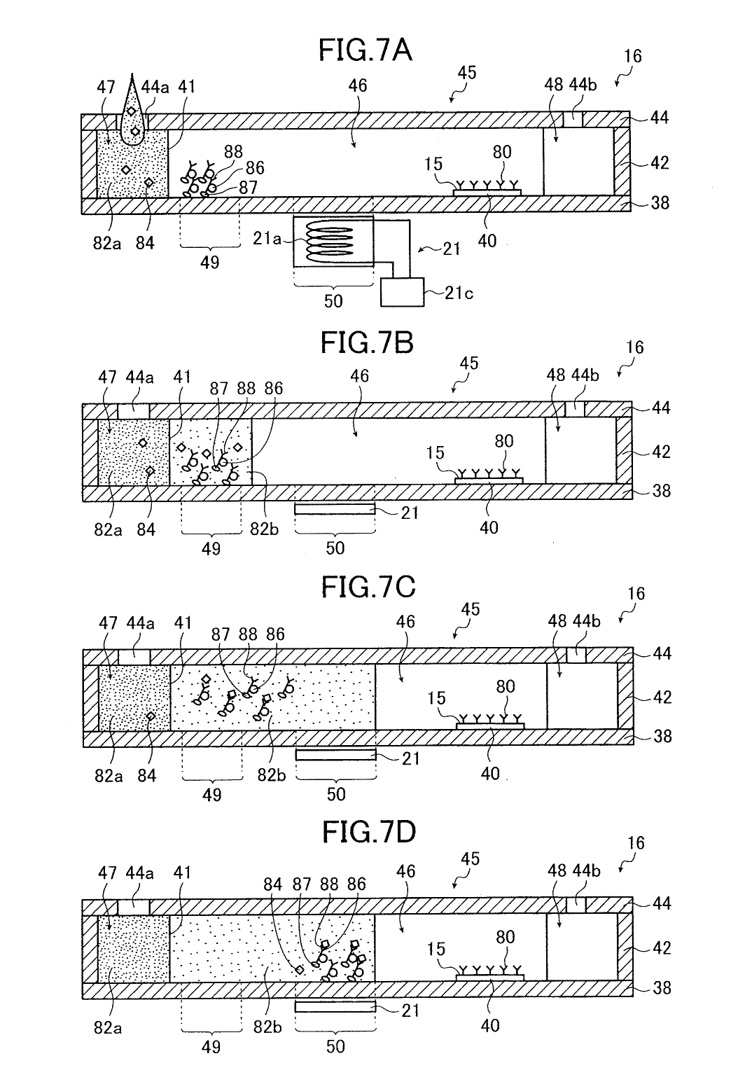

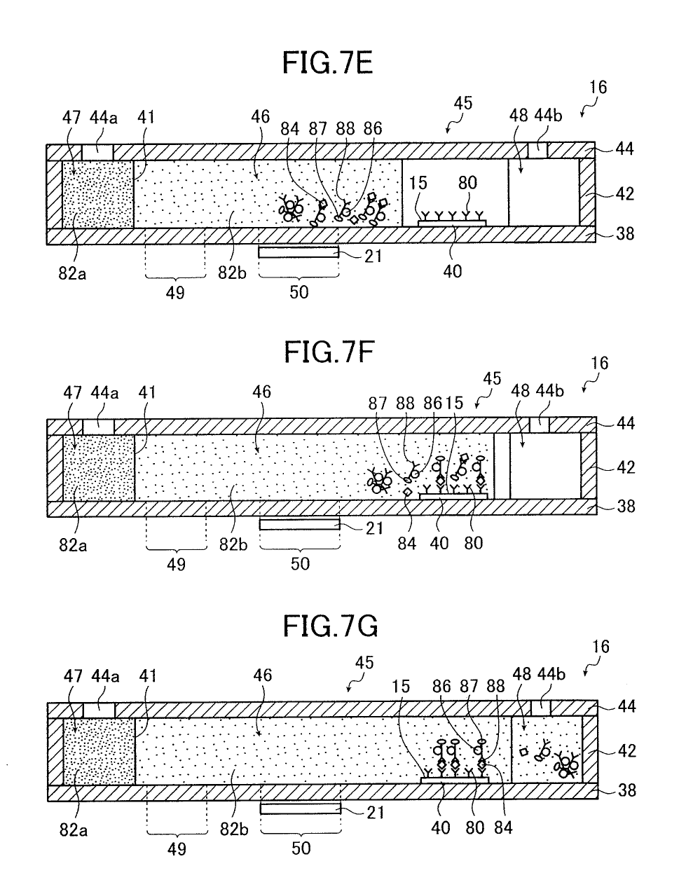

FIGS. 6A to 6E and 7A to 7G illustrate how the liquid sample flows in the inspection chip 16. FIGS. 6A to 6E are top plan views illustrating different states of the inspection chip 16 and FIGS. 7A to 7G are longitudinal cross sections illustrating different states of the inspection chip 16.

While the following description is made of a case where the liquid sample fed into the inspection chip 16 is blood 82a as a representative example, the liquid sample may be any specimen, such as urine, used for immunoassay using antigen-antibody reaction.

Although the immunoassay typically uses the sandwich technique or the competition technique, both measuring methods may be suitably used in the present invention. Here, an example using the sandwich technique will be described; the competition technique will be described later in detail.

According to the sandwich technique, a sandwich structure like “first antibody/analyte/second antibody” is fabricated, and the analyte in the liquid sample is quantitatively measured based on the quantity of the sandwich structure.

According to the sandwich technique, two or more molecules of antibody need to bind to the analyte and therefore two or more epitopes are required. Thus, when the analyte has a small molecular weight, measurement may be impossible.

As illustrated in FIGS. 6A and 7A, the blood (whole blood) 82a containing the analyte 84 is dropped from the feed inlet 44a of the cover 44 of the inspection chip 16 into the leading end portion 47 of the channel 45 of the channel substrate 42.

The blood 82a dropped into the leading end portion 47 is filtered by the blood cell filter 41, which passes plasma 82b and filters off red blood cells, white blood cells, etc. The plasma 82b then moves because of the capillary shape through the tube formed by the linear portion 46 and the transparent glass cover 44 toward the terminal end portion 48.

Next, the suction unit 22 is attached to the terminal end portion 48 as illustrated in FIG. 5 to suck the plasma 82b in the channel 45 from the terminal end portion 48. The suction unit 22 is not shown in FIGS. 6A to 6E and FIGS. 7A to 7G.

The plasma 82b moving from the leading end portion 47 of the channel 45 through the linear portion 46 of the channel 45 toward the terminal end portion 48 reaches the secondary antibody deposit region 49 in the linear portion 46 as illustrated in FIGS. 6B and 7B. When the plasma 82b reaches the secondary antibody deposit region 49, an antigen-antibody reaction occurs between the analyte 84 contained in the plasma 82b and the secondary antibody 88 placed in the secondary antibody deposit region 49, whereupon the analyte 84 and the secondary antibody 88 bind to each other.

Since the secondary antibody 88 is labeled by the fluorescent substance 86, the analyte 84 bound to the secondary antibody 88 is labeled by the fluorescent substance 86. Since the secondary antibody 88 is labeled by the magnetic particles 87, the analyte 84 bound to the secondary antibody 88 is labeled by the magnetic particles 87.

As illustrated in FIGS. 6C and 7C, due to a suction operation with the pump 53, the plasma 82b, after passing through the secondary antibody deposit region 49, moves further on along the linear portion 46 toward the terminal end portion 48 and reaches the concentration region 50.

When the liquid sample 82 reaches the concentration region 50, a given level of current is passed through the electric coil 21a of the concentration unit 21 to produce magnetic field in the concentration region 50 as illustrated in FIG. 7D. In this state, the pump 53 is activated to perform suction so that the plasma 82b is caused to flow downwards, whereupon magnetic force directed toward the bottom surface of the inspection chip 16 acts upon the magnetic particles 87, causing the analyte 84 to move toward the bottom surface of the inspection chip 16 for concentration.

When a given quantity of the plasma 82b has flowed downwards, the supply of electric current to the electric coil 21a is stopped, completing the concentration process.

This concentration process permits reduction of time for a reaction that is to follow in which the analyte 84 bound to the secondary antibody 88 is caused to react with the primary antibody 80 located on the sensing surface 15 of the metal film 40.

Upon completion of the concentration, suction by the pump 53 causes the plasma 82b to flow further on along the linear portion 46 toward the terminal end portion 48 as illustrated in FIGS. 6D, 7E and 7F.

As illustrated in FIG. 7E, the secondary antibody 88 labeled by the fluorescent substance 86 and the magnetic particles 87, the secondary antibody 88 further having the analyte bound thereto, and aggregates of the secondary antibody 88 are caused to flow toward the sensing surface 15 in this order, i.e., in increasing order of weight.

The concentration effected by the concentration unit 21 may cause pieces of the secondary antibody 88 to gather together to form aggregates each having a large mass.

Taking advantage of the fact that the speed at which to arrive at the sensing surface 15 of the metal film 40 varies with the mass, the aggregates can be prevented from binding to the sensing surface by stopping the flow of the plasma 82b or by completing the measurement before the aggregates reach the sensing surface 15.

In the present invention, therefore, it is preferable to provide a control means (not shown) for controlling the concentration unit 21 and the suction unit 22 in order to prevent the aggregates formed in the concentration region 50 located upstream of the sensing surface 15 of the inspection chip 16 from reaching the sensing surface 15.

The control means preferably controls the pump 53 to cause the plasma 82b in the channel 45 to flow while controlling the concentration unit 21 so as to retain only the aggregates formed in the concentration region 50 are retained onto the wall surface of the concentration region 50. Alternatively, the control means preferably controls the pump 53 so that the flow of the plasma 82b is stopped before the aggregates reach the sensing surface 15 as described earlier. Alternatively, the control means preferably also controls the light source driver 26 and the light detection unit 18 as well as the pump 53 and the concentration unit 21 so that the measurement of the analyte 84 is completed before the aggregates reach the sensing surface 15 as described earlier.

When the plasma 82b reaches the sensing surface 15 of the metal film 40, an antigen-antibody reaction occurs between the analyte 84 contained in the plasma 82b and the primary antibody 80 secured to the sensing surface 15, and the analyte 84 is captured by the primary antibody 80 as illustrated in FIG. 7F. Since the analyte 84 captured by the primary antibody 80 is labeled by the fluorescent substance 86 in the secondary antibody deposit region 49, the primary antibody 80 that has captured the analyte 84 becomes labeled by the fluorescent substance 86. In other words, the analyte 84 becomes sandwiched between the primary antibody 80 and the secondary antibody 88.

As the pump 53 performs suction, the plasma 82b, after passing through the sensing surface 15 of the metal film 40, moves further on to the terminal end portion 48 as illustrated in FIGS. 6E and 7G. The analyte 84 that is not captured by the primary antibody 80 and the secondary antibody 88 and the fluorescent substance 86 to which the analyte 84 is not bound also move to the terminal end portion 48 along with the plasma 82b.

In the process of concentrating the plasma 82b, even when no aggregate of the secondary antibody 88 is formed, a so-called non-specific adsorption may occur whereby the secondary antibody 88 labeled by the fluorescent substance 86 binds to the primary antibody 80 on the metal film 40 without binding to the analyte 84, possibly making accurate quantitative measurement of the analyte 84 impossible. However, a planned reaction can be accomplished in a short period of time by causing the secondary antibody 88 labeled by the fluorescent substance 86 to flow quickly across the metal film 40 by suction performed by the pump 53.

When there remains on the metal film 40 only the sandwich structure formed of the secondary antibody 88 labeled by the fluorescent substance 86, the analyte 84, and the primary antibody 80, the metal film 40 is irradiated with excitation light.

Specifically, the light source 12 emits excitation light according to the electric current supplied from the light source driver 26 based upon the modulation signal produced by the FG 24. The excitation light emitted from the light source 12 passes through the incidence optics 14. Specifically, the excitation light is collimated by the collimator lens 30, condensed by the cylindrical lens 32 in only one direction, and polarized by the polarizing filter 34. Preferably, a spectrum adjusting means is provided between the cylindrical lens 32 and the polarizing filter 34 to achieve uniformity in intensity among rays of light in a given wavelength range.

The light passing through the incidence optics 14 enters the prism 38 through one of its faces, hits the boundary surface between the prism 38 and the metal film 40, and is totally reflected by the metal film 40 before being emitted through the other plane of the prism 38. The cylindrical lens 32 focuses the light to a focal point a given distance away from and beyond the boundary surface between the prism 38 and the metal film 40.

As mentioned above, the parallel light produced by the collimator lens 30 is condensed by the cylindrical lens 32 in only one direction so that the excitation light may hit the boundary surface between the prism 38 and the metal film 40 at the same angle in a direction parallel to the direction in which the linear portion 46 extends.

With the total reflection of the excitation light, evanescent light exudes on the sensing surface 15 (the surface opposite from the prism 38) of the metal film 40 and excites surface plasmons in the metal film 40. The surface plasmons in turn produce an electric field distribution on the surface of the metal film 40, forming enhanced field regions.

Then, the surface plasmons resonate with the evanescent waves generated by the excitation light hitting the boundary surface between the prism 38 and the metal film 40 at a specific angle meeting the plasmon resonance conditions or the excitation light hitting the boundary surface at said specific angle from among the excitation light hitting the boundary surface at angles within a given range, thus producing surface plasmon resonances (plasmon field enhancement effect). Thus, further intensified field enhancement is achieved in the areas where surface plasmon resonances (plasmon field enhancement effect) have been produced. The condition for plasmon resonances to occur is that the wavenumber vector of the evanescent waves generated by the incident light is equal to the wavenumber of surface plasmons to establish a wavenumber match. As mentioned above, this plasmon resonance condition depends on various factors including the type of the sample, its state, the thickness of the metal film, its density, the wavelength of the excitation light, and its incident angle.

The plasmon resonance angle and the incident angle of the excitation light (its rays) are the angle it forms with the line normal to the sensing surface of the metal film.

When the fluorescent substance 86 is present in areas where the evanescent waves have exuded, the fluorescent substance 86 is excited to generate fluorescence. This fluorescence is enhanced by the effect produced by the surface plasmons that are present in substantially the same areas as those where the evanescent waves have exuded, particularly the effect produced by the field enhancement that has been intensified by the surface plasmon resonances. The fluorescent substance outside the areas where the evanescent waves have exuded is not excited and hence does not generate fluorescence.

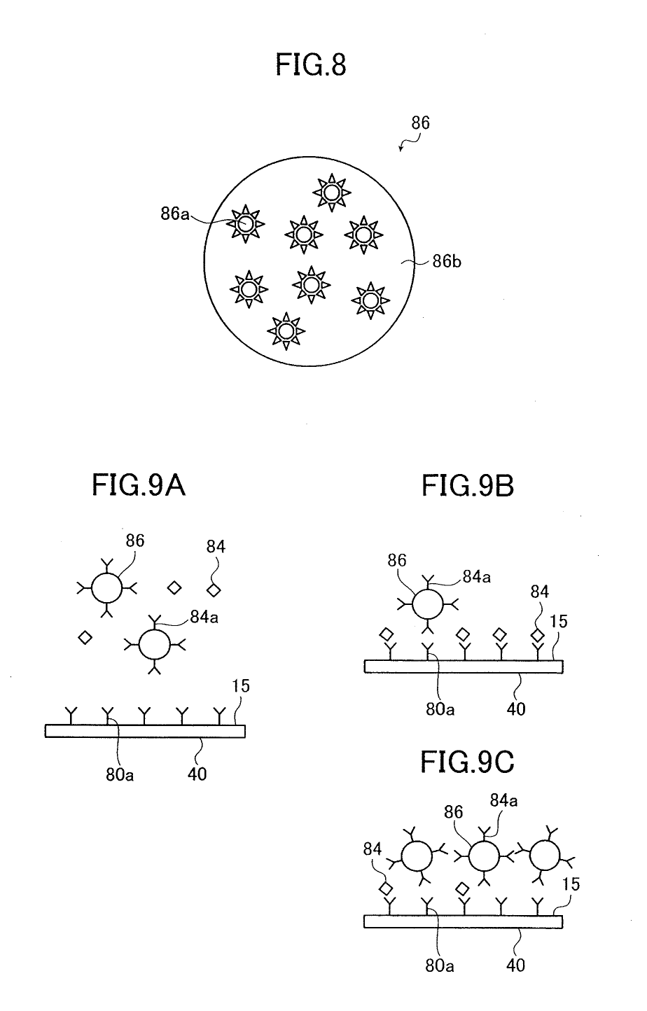

When the fluorescent substance 86 in the liquid sample is too close to the metal film 40, the energy excited within the fluorochrome can transit to the metal film 40 before generating fluorescence, resulting in metal quenching or failure to generate fluorescence. Therefore, the fluorescent substance 86 used in the present invention preferably comprises fluorochrome molecules 86a and a quenching prevention portion 86b holding therein the fluorochrome molecules 86a as illustrated in FIG. 8. The quenching prevention portion 86b transmits fluorescence emitted from the fluorochrome molecules 86a and prevents the fluorochrome molecules 86a from coming too close to the metal film 40. Having such a configuration, the fluorescent substance 86 possesses a quenching prevention capability.

Using the fluorescent substance 86 having such a quenching prevention capability, a certain distance can be secured between the metal film 40 and the fluorochrome molecules without the need to provide a SAM film or a CMD film on the metal film 40 for metal quenching prevention. Thus, the metal quenching can be prevented effectively by a simple method.

Since the fluorescent substance 86 illustrated in FIG. 8 contains a plurality of fluorochrome molecules 86a, the amount of fluorescence emitted can be greatly increased as compared with a case where the fluorochrome molecules 86a are used for labeling.

The diameter of the fluorescent substance 86 is preferably 5300 nm or less, more preferably 70 nm to 900 nm both inclusive, and most preferably 130 nm to 500 nm both inclusive.

The quenching prevention portion 86b may be formed, for example, of polystyrene or SiO₂ but is not limited specifically, provided that it can contain therein the fluorochrome molecules 86a, transmit and emit the fluorescence generated by the fluorochrome molecules 86a to the outside, and prevent metal quenching of the fluorochrome molecules 86a.

Thus, the fluorescent substance 86 labeling the analyte 84 secured to the metal film 40 is excited to generate fluorescence.

The light emitted from the fluorescent substance 86 enters the first lens 66 of the light detection unit 18, passes through the cut-off filter 68, is condensed by the second lens 70, and enters the PD 62 where it is converted into an electric signal. That component of the light that is incident on the first lens 66 and which has the same wavelength as the excitation light cannot pass through the cut-off filter 68 and thus cannot reach the PD 62.

The electric signal generated in the PD 62 is amplified as detection signal by the PD 64 and thence fed to the lock-in amplifier 74 that amplifies the signal component that is synchronous with the reference signal supplied from the FG 24. Thus, the light caused by the excitation light can be sufficiently amplified to permit sure distinction between the light emitted from the fluorescent substance 86 on the one hand and light that enters the PD 62 from other than the light detection unit 60 such as, for example, light from fluorescent lamps in the room, etc. as well as noise components caused by dark currents generated in the PD 62 on the other hand.

The detection signal amplified by the lock-in amplifier 74 is sent to the PC 76.

The PC 76 detects the detection signal to an analog signal, and detects the concentration of the analyte 84 in the plasma 82b from the result of computation on the analyte 84 based on a preliminarily stored calibration curve.

Thus, the mass and the concentration of the analyte 84 in the plasma 82b are detected.

According to the sensing device 10 of this embodiment, the time required for the reaction between the antigen (analyte 84) and the antibody 80 secured to the sensing surface 15 can be reduced, and still the antigen can be bound to the antibody sufficiently.

Specifically, before allowing the liquid sample 86 to come into contact with the sensing surface 15 (metal film 40), the concentration unit 21 concentrates the liquid sample 86 containing the analyte 84 toward the sensing surface 15, and the suction unit 22 causes the thus concentrated liquid sample 86 to be fed to the sensing surface 15, thereby completing the antigen-antibody reaction in a short period of time and measurement of the analyte 84 with a high sensitivity.

According to the sensing device 10 of this embodiment, the aggregates can be prevented from binding to the sensing surface 15, and measurement can be performed with a further increased sensitivity by stopping the flow of the liquid sample 86 or by completing the measurement before the aggregates reach the sensing surface 15.

This also eliminates the need to provide the concentration unit 21 on the sensing surface 15 and hence enables measurement of the analyte 84 without using costly transparent electrodes.

Next, the effects and operations of the sensing device 10 using the competition technique to implement immunoassay will be described.

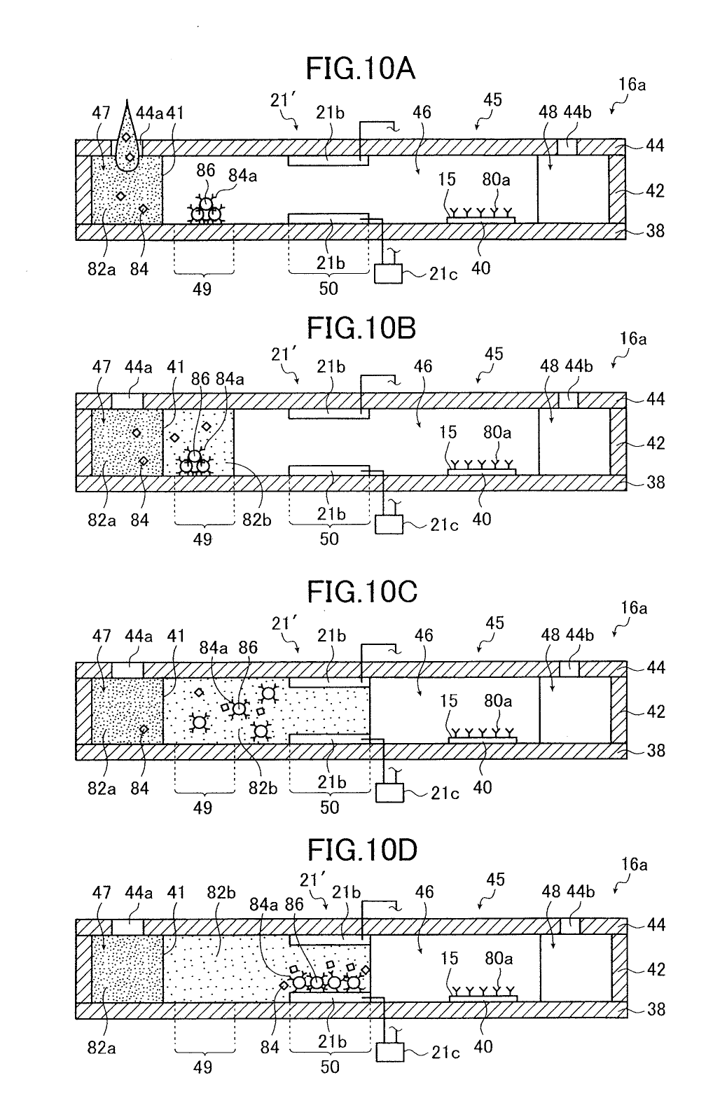

As illustrated in FIG. 9A, the antigen 84a labeled by the fluorescent substance 86 and having the same immune response as the analyte 84 needs to be provided. A primary antibody 80a that can specifically bind to both the analyte 84 and the antigen 84a is previously secured to the sensing surface 15 of the metal film 40. The antigen 84a labeled by the fluorescent substance 86 is mixed with the analyte 84 in a given concentration and subjected competitively to antigen-antibody reaction with the primary antibody 80a secured to the metal film 40.

As a result of the reaction, when the analyte 84 is highly concentrated, a reduced quantity of the antigen 84a binds to the primary antibody 80a and there are a reduced number of pieces of the fluorescent substance 86 on the metal film 40, as illustrated in FIG. 9B, so that the fluorescent intensity decreases.

By contrast, when the analyte 84 is in low concentration, a greater quantity of the antigen 84a binds to the primary antibody 80a and there are an increased number of pieces of the fluorescent substance 86 on the metal film 40, as illustrated in FIG. 9C, increasing the fluorescent intensity.

Thus, the competition technique, in which a single epitope in the analyte suffices to enable measuring, is suitable to detection of a low-molecular-weight substance.

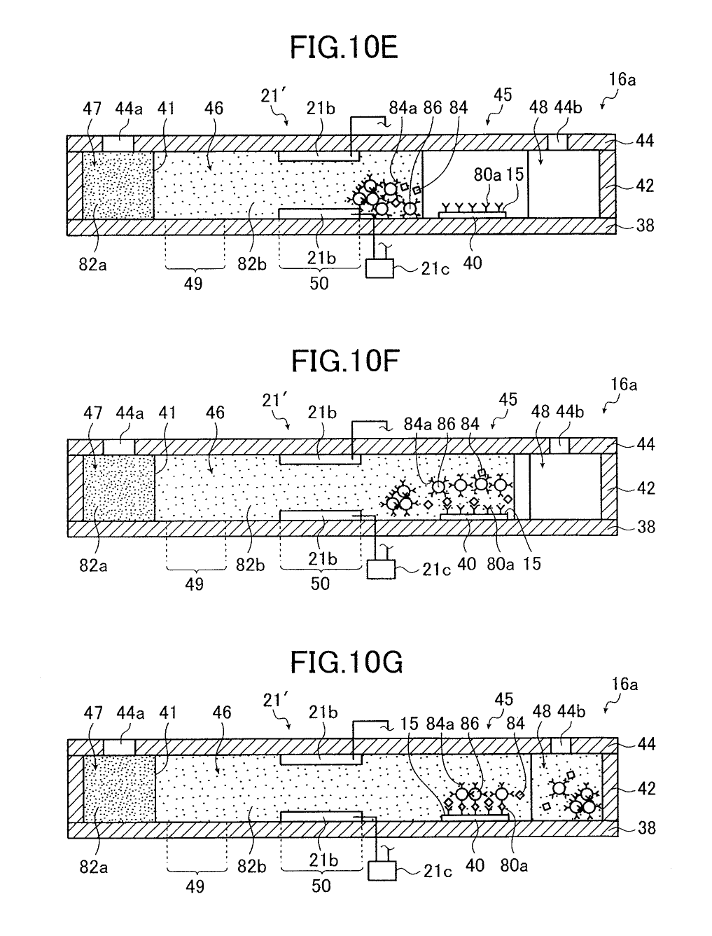

Next, the effects and operations of the sensing device using the competition technique will be described referring to FIGS. 10A to 10G. Note that the fluorescent detection method, which is comparable to the sandwich technique, will not be described.

Where the competition technique is used, the sensing device 10 illustrated in FIG. 1 uses an inspection chip 16a illustrated in FIG. 10A instead of the inspection chip 16.

The inspection chip 16a has substantially the same configuration as the inspection chip 16 illustrated in FIG. 4 except that the former has the antigen 84a, which is labeled by the fluorescent substance 86, placed in the secondary antibody deposit region 49 instead of the secondary antibody 88 labeled by the fluorescent substance 86 and the magnetic particles 87 and has the primary antibody 80a secured to the sensing surface 15 instead of the primary antibody 80. The concentration unit 21′ illustrated in FIG. 10A has substantially the same configuration as the concentration unit 21 illustrated in FIG. 4 except that the former has electrodes 21b, 21b for generating electric fields instead of the electromagnetic coil 21a for generating magnetic fields, and the electrodes 21b, 21b are disposed opposite the bottom surface and the cover 44 of the linear portion 46 of the channel 45 in the concentration region 50.

While the following description is made of a case where the liquid sample fed into the inspection chip 16a is blood 82a as a representative example, the liquid sample may be any specimen, such as urine, used for immunoassay using antigen-antibody reaction.

The suction unit illustrated in FIG. 5, not shown in FIGS. 10A to 10G, is connected to the terminal end portion 48.

First, as illustrated in FIG. 10A, the blood (whole blood) 82a containing the analyte 84 is dropped from the feed inlet 44a of the cover 44 of the inspection chip 16a into the leading end portion 47 of the channel 45 of the channel substrate 42.

The blood 82a dropped into the leading end portion 47 is filtered by the blood cell filter 41 to remove red blood cells and white blood cells, allowing plasma 82b to pass through the filter. The plasma 82b then moves by capillary action through the tubular channel 45 formed by the linear portion 46 and the cover 44 toward the terminal end portion 48.

Next, the suction unit sucks the plasma 82b in the channel 45 from the terminal end portion 48.

As illustrated in FIG. 10B, when the plasma 82b reaches the secondary antibody deposit region 49 of the linear portion 46, the analyte 84 contained in the plasma 82b mixes with the antigen 84a labeled by the fluorescent substance 86 placed in the secondary antibody deposit region 49.

As illustrated in FIG. 100, the plasma 82b, after passing through the secondary antibody deposit region 49, is caused to move further on through the linear portion 46 toward the terminal end portion 48 by suction effected with the suction unit and reaches the concentration region 50.

When the plasma 82b reaches the concentration region 50, a voltage is applied from an electric source 21c to the electrodes 21b, 21b of the concentration unit 21′ to produce electric field as illustrated in FIG. 10D. In this state, suction effected by the suction unit causes the plasma 82b to flow downwards, whereupon Coulomb's force is generated according to the electric charges of the analyte 84 and the antigen 84a and acts to move the analyte 84 and the antigen 84a toward the bottom surface of the inspection chip 16a, achieving concentration of the plasma 82b.

When a given quantity of the plasma 82b has flowed downwards, application of the electric voltage to the electrodes 21b, 21b of the concentration unit 21′ is stopped to terminate the concentration process.

This concentration process permits reduction of time for a reaction that is to follow in which the analyte 84 and the antigen 84a are caused to react with the primary antibody 80a located on the sensing surface 15 of the metal film 40.

Note that the antigen 84a labeled by the fluorescent substance 86 may gather together to form aggregates as in the sandwich technique.

Upon completion of the concentration, suction by the suction unit causes the plasma 82b to flow further on through the linear portion 46 of the channel 45 toward the terminal end portion 48 as illustrated in FIGS. 10E and 10F.

As illustrated in FIG. 10E, the analyte 84, the antigen 84a labeled by the fluorescent substance 86, and the aggregates of the antigen 84a are caused to flow toward the sensing surface 15 in this order, i.e., in increasing order of weight.

Because the speed with which to arrive at the sensing surface 15 of the metal film 40 varies with the mass, a control means (not shown) is preferably provided to control the concentration unit 21′, the suction unit, etc., to ensure that the aggregates do not reach the sensing surface 15. Using this control means to stop the flow of the plasma 82b or complete the measurement before the aggregates reach the sensing surface 15, the aggregates can be prevented from binding to the sensing surface 15.

When the plasma 82b reaches the sensing surface 15 of the metal film 40, an antigen-antibody reaction occurs between the analyte 84 contained in the plasma 82b and the antigen 84a on the one hand and the primary antibody 80a secured to the sensing surface 15 on the other hand, and the analyte 84 and the antigen 84a are captured by the primary antibody 80a as illustrated in FIG. 10F.

After passing through the sensing surface 15, the plasma 82b is caused by the suction effected by the suction unit to move further on to the terminal end portion 48 as illustrated in FIG. 10G. The analyte 84 and the antigen 84a that are not captured by the primary antibody 80a also move to the terminal end portion 48 along with the plasma 82b.

Thus, also the competition technique permits detection of the concentration of the analyte 84 by binding the analyte 84 and the antigen 84a labeled by the fluorescent substance 86 to the sensing surface 15 of the metal film 40, and then performing fluorescence detection in the same manner as in the sandwich technique.

While, in the illustrated example, an electric field is generated by the concentration unit 21′ having the electrodes 21b, 21b to achieve concentration of the antigen 84a, the antigen 84a may be previously labeled by such a labeling substance for concentration that increases Coulomb's force acting upon the antigen 84a by means of electric field. Further, the fluorescent substance 86 labeling the antigen 84a may be previously labeled by such a labeling substance for concentration that increases Coulomb's force acting upon the fluorescent substance 86 by means of electric field.

Another alternative is that the fluorescent substance 86 is formed of fluorescent magnetic beads and the antigen 84a is labeled by the fluorescent magnetic beads to generate magnetic field using the concentration unit 21 having the magnetic coil 12a as illustrated in FIG. 4.

### Embodiment 2

While Embodiment 1 uses the evanescent excitation illumination method whereby evanescent waves are generated on the surface of the metal film to detect the detection light produced by evanescent excitation, the invention is not limited this way. The epi-illumination method may be used whereby light emitted from the light source is directly admitted through the transparent cover of the inspection chip to produce fluorescence from the sensing surface.

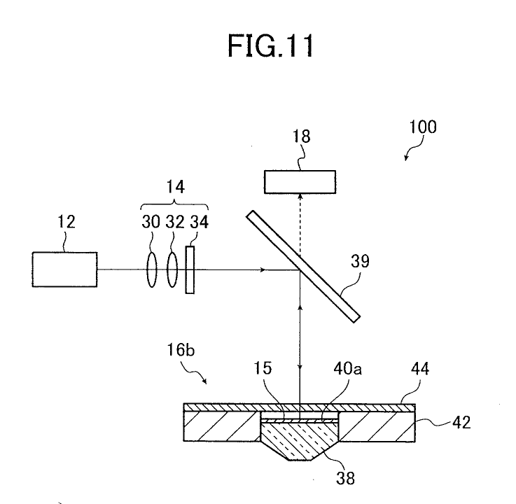

FIG. 11 illustrates a sensing device 100 according to Embodiment 2 using the epi-illumination method.

The sensing device 100 has the same configuration as the sensing device 10 according to Embodiment 1 illustrated in FIG. 3 except that in the former device, the light emitted from the light source 12 is admitted through the transparent cover 44 by reflecting the light with a half-mirror 39 instead of admitting the light through the prism 38 of the inspection chip 16, and that the former uses an inspection chip 16b comprising a metal layer 40a instead of the inspection chip 16 comprising the metal film 40.

The half-mirror 39 reflects the light emitted from the light source 12 and transmitted by the incidence optics 14 to cause the light to hit the sensing surface 15 forming the surface of the metal layer 40a from above the transparent cover 44 of the inspection chip 16b at right angles. The half-mirror 39 also transmits the fluorescence occurring from the sensing surface 15 and allows it to enter the light detection unit 18.

The metal layer 40a comprises a metal microstructure having a textured surface structure in which the difference in height between the high and low points is smaller than the wavelength of the excitation light or metal nano-rods each smaller than the wavelength of the excitation light.

The metal microstructure or the metal nano-rods used as the metal layer 40a is preferably formed of a substance containing as a major component at least one kind of metal selected from the group consisting of Au, Ag, Cu, Al, Pt, Ni, Ti, and an alloy thereof.

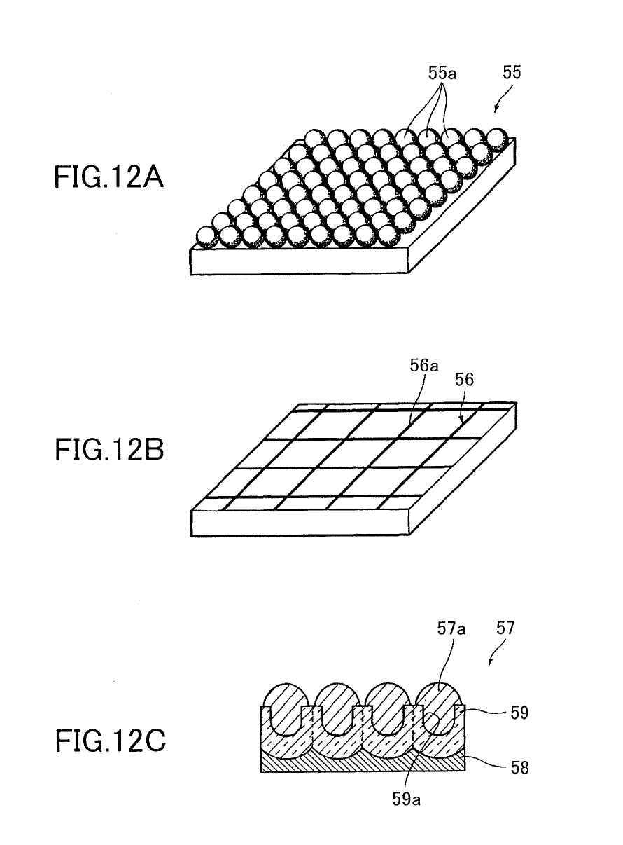

FIGS. 12A to 12C illustrate specific examples of metal layer 40a.

The metal layer 40a illustrated in FIG. 12A is formed of a metal microstructure 55 comprising arrays of metal particles 55a fixed to one surface of the prism 38 made of a dielectric material. The metal particles 55a may be arranged in any appropriate pattern but is preferably arranged in a substantially regular pattern. With such a configuration, an average diameter and pitch of the metal particles 55a are designed to be smaller than the wavelength of the excitation light.

The metal layer 40a illustrated in FIG. 12B is configured by a metal microstructure 56 formed of a patterned metal layer in which fine metal lines 56a are patterned in the form of a grid. The patterned metal layer may be designed with an appropriate pattern as desired but the pattern is preferably a substantially regular pattern. With such a configuration, an average line width and pitch of the fine metal lines 56a are designed to be smaller than the wavelength of the excitation light.

The metal layer 40a illustrated in FIG. 12C is configured by a metal microstructure 57 formed of metal members 57a each in the form of a mushroom grown in a micropore 59a made of a metal oxide 59 formed in a process of anodizing a metal substance 58 such as aluminum (Al), as described in JP 2007-171003 A.

The metal oxide 59 corresponds to the prism 38. The metal microstructure 57 may be obtained by anodizing a part of a metal substance (e.g. Al) to produce a metal oxide (e.g. Al₂O₃) and plating or otherwise treating the metal members 57a to grow them in the micropores 59a of the metal oxide 59 formed in the anodizing process.

In the example illustrated in FIG. 12C, the top of each mushroom-shaped metal member 57a is in the form of a particle such that, as seen from the surface of the metal oxide 59, the metal microstructure 57 seems to have a structure formed of arrayed metal particles. With such a configuration, the top of each mushroom-shaped metal member 57a provides a projection whose average diameter and pitch are designed to be smaller than the wavelength of the measuring light.

The metal layer 40a for producing localized plasmons in response to the excitation light may be any of various types of metal microstructures besides the above metal microstructure including those described in, for example, JP 2006-322067 A and JP 2006-250924 A using a microstructure obtained by anodizing a metal substance.

The metal layer 40a capable of generating localized plasmons may be formed of a metal film having a roughened surface. The surface roughening may be achieved by an electrochemical method and the like using, for example, oxidoreduction.

Alternatively, the metal layer 40a may be configured by metal nano-rods disposed on the prism 38. The minor axis of the metallic nano-rods measures about 3 nm to 50 nm, and the major axis measures about 25 nm to 1000 nm. The major axis is smaller than the wavelength of the excitation light. For details of the configuration of metallic nano-rods, reference may be had, for example, to JP 2007-139612 A.

In the sensing device 100, the excitation light is caused to irradiate the surface of the metal layer 40a formed by the metal structure or the metal nano-rods to excite surface plasmons and produce localized plasmon resonances, generating enhanced electric fields, where intensified fluorescence is measured.

Because the sensing device 100 is provided with the metal layer 40a capable of producing localized plasmon resonance as described above, fluorescence can be measured without meeting the condition of total reflection of the excitation light at the interface between the metal film 40 and the prism 38 as in Embodiment 1.

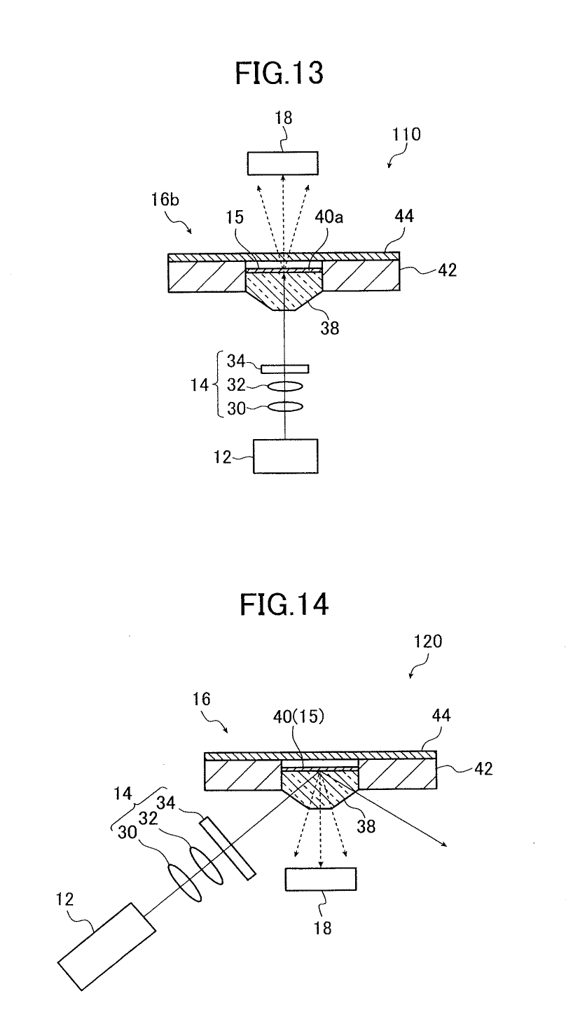

FIG. 13 illustrates a sensing device 110 according to a variation of Embodiment 2.

The sensing device 110 uses an illumination method whereby a metal microstructure or the metal layer 40a formed of metal nano-rods is irradiated with light emitted from the light source 12 (excitation light) at right angles from below.

Such a configuration, provided with the metal layer 40a capable of producing localized plasmon resonance, also enables measuring of fluorescence as with the sensing device 100 illustrated in FIG. 11.

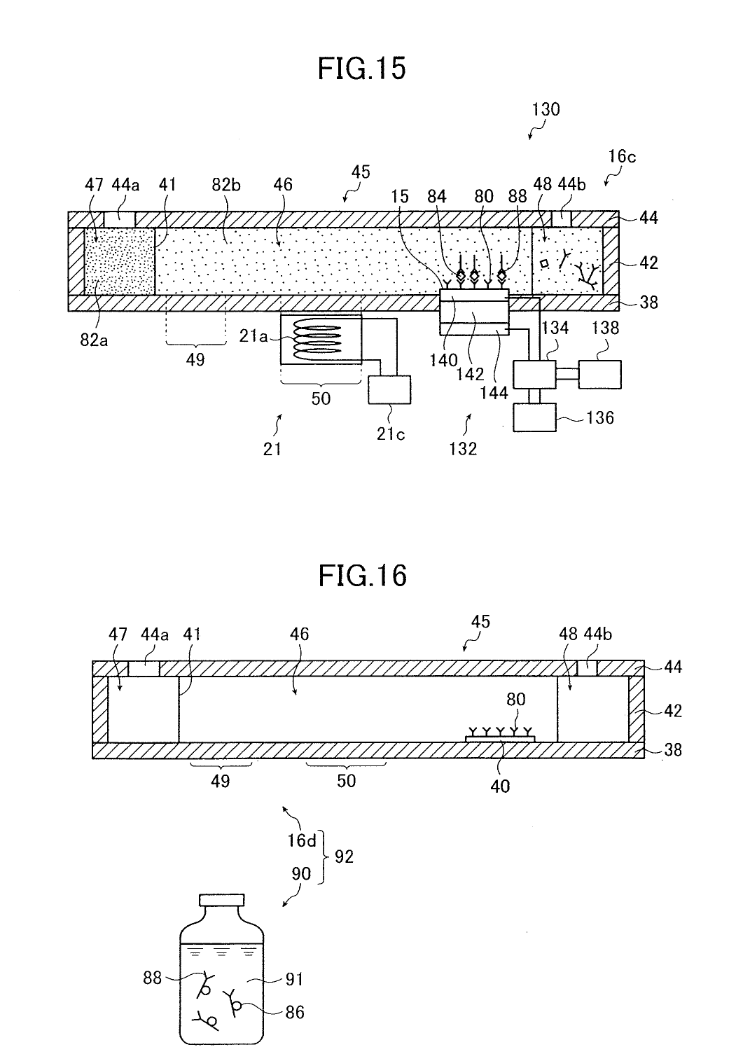

FIG. 14 illustrates a sensing device 120 according to another variation of Embodiment 2.

The sensing device 120 detects the radiation light with the light detection unit 18 emitted from that surface of the metal film 40 opposite from that in the sensing device 10 illustrated in FIG. 3, i.e., the surface of the metal film 40 facing the prism 38.

Accordingly, it is not the fluorescence generated by the fluorescent substance 86 that is detected; by a method employed here, the radiation light generated when fluorescence newly excites surface plasmons on the metal film 40 (SPCE or surface plasmon-coupled emission) is detected on the side where the prism 38 is provided.

According to the SPCE method, the light detection unit 18 is disposed in a position such that it detects radiation light leaving the same surface that is hit by the excitation light.

When the liquid sample used is the whole blood, the whole blood causes light absorption in fluorescence measuring described earlier, and this necessitates pretreatment whereby the blood is filtered by the blood cell filter 41 to reduce the blood to plasmatic blood components. The SPCE method eliminates the need of such a pretreatment so that the blood can be used as it is.

Thus, the invention may be applied to sensing devices implementing a variety of illumination methods and fluorescence detection methods using a fluorescent substance as an optical detection labeling substance for labeling a secondary antibody. The invention is not limited this way, however. Even where the secondary antibody is not labeled by the fluorescent labeling substance, the invention permits sensing using the surface plasmon resonance illumination method, i.e., a substance detection method, whereby light is admitted and directed to hit the metal film 40 of the inspection chip 16a or the metal layer 40a of the inspection chip 16b at a given incident angle at which surface plasmons are generated, to detect the scattered light of the generated surface plasmons, Raman scattered light, and other like scattered light.

One may also use a detection method using a scattering enhancement labeling substance as optical detection labeling substance for intensifying the scattered light of the generated surface plasmons, Raman scattered light, and other like scattered light.

Now, the principle of the surface plasmon resonance illumination method will be described.

Excitation light allowed to hit the metal film 40 at an angle at which surface plasmons are generated results in excitation of surface plasmons in the metal film 40.

When the wavenumber of the excitation light hitting the boundary surface between the prism 38 and the metal film 40 at a specific angle meeting the plasmon resonance conditions coincides with the frequency of the surface plasmons to establish a wavenumber match, surface plasmon resonances are generated, causing free electrons of the metal film 40 to vibrate strongly.

In the presence of surface plasmon resonances, the vibrations of free electrons of the metal film 40 generate electric fields outside the metal film 40. The fluorescent substance 86 present in the electric fields is excited and generates fluorescence.

The evanescent illumination method and the surface plasmon resonance method differ in that the evanescent waves do not mediate the generation of fluorescence in the latter.

When surface plasmon resonances occur, most of the optical energy changes to surface plasmons of the metal film 40 so that the intensity of the light reflected by the metal film 40 becomes 0.

Because the received optical energy creates electric fields concentrated close to the metal film 40 (field enhancement region), further intensified field enhancement is achieved in the regions where surface plasmon resonances (plasmon field enhancement effect) have been produced. Thus, the fluorescent substance 86 in these electric fields is excited and generates fluorescence, and the intensity of the fluorescence is enhanced.

The fluorescence enhanced by the surface plasmon resonance illumination method has a higher fluorescence intensity than those obtained by the evanescent illumination method and the epi-illumination method. This is because by the evanescent illumination method, the evanescent waves exuded upon total reflection at the interface between the metal film 40 and the prism 38 only excite fluorescence to produce light, and by the epi-illumination method, most of the illumination light only passes by without hitting the fluorescent substance 86.

The magnetic particles used as concentration labeling substance may be used as optical detection labeling substance to detect scattering of magnetic particles. Alternatively, gold colloid may be used to detect scattering of gold colloid. According to the invention, the secondary antibody bound to the analyte is concentrated, and therefore labeling is not used. Thus, the invention may be applied to a scattering method where the sensitivity is low and hence the detection is not easy, making it possible to apply the scattering method to detection of a substance.

### Embodiment 3

Measuring may be achieved using the quartz crystal microbalance (QCM) measuring method other than the above-described substance detection method using optical detection.

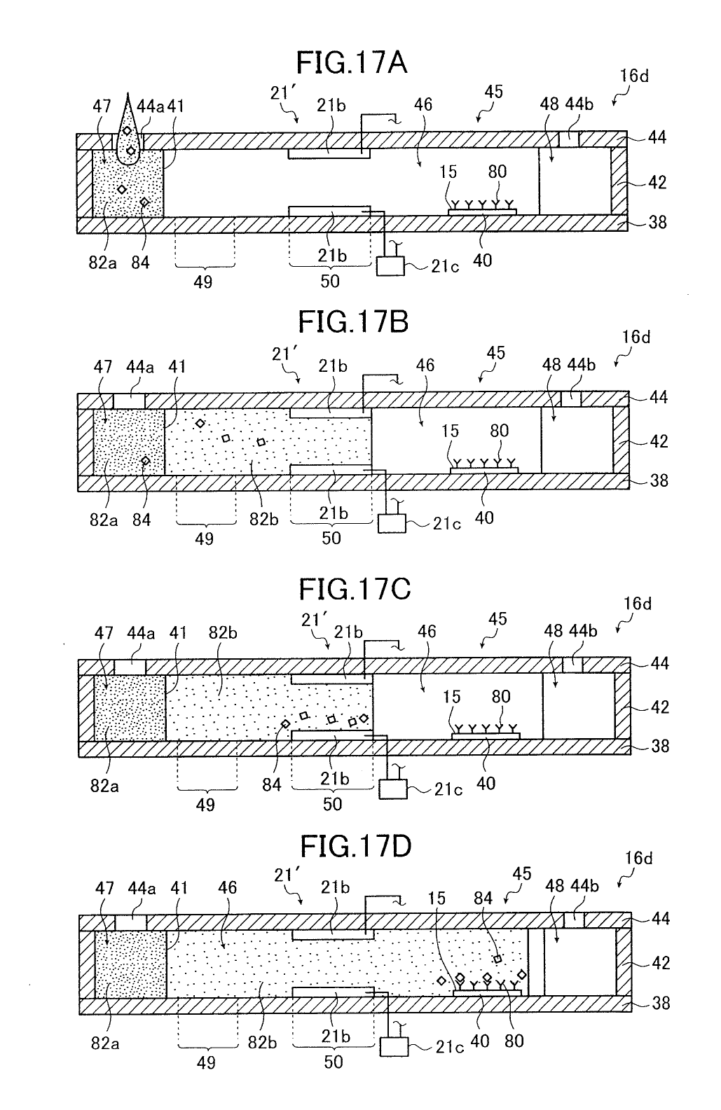

FIG. 15 illustrates a sensing device 130 according to Embodiment 3 of the invention.

The sensing device 130 has the same configuration as the sensing device 10 according to Embodiment 1 illustrated in FIGS. 1 to 5 except for the detection means. Therefore, like characters in the drawings represent like components, and description thereof will not be repeated, concentrating mainly on the differences.

The sensing device 130 uses a QCM sensor 132 for implementing the QCM measuring method. The QCM sensor 132 is so disposed in relation to an inspection chip 16c that the detection surface of the QCM sensor 132 forms the sensing surface 15 of the inspection chip 16c. The sensing device 130 further comprises the concentration unit 21 including the electromagnetic coil 21a disposed for the concentration region 50, an oscillation circuit 134 for driving the QCM sensor 132 with a given resonant frequency, an electric power supply 136 for supplying electric power to the oscillation circuit 134, and a QCM analyzer 138 connected to the oscillation circuit 134. The QCM analyzer 138 detects the binding quantity of the analyte (antigen) 84 bound to the primary antibody 80 secured to the sensing surface 15 in the inspection chip 16c according to the variation in resonant frequency detected by the QCM sensor 132.

The inspection chip 16c only differs from the inspection chip 16 illustrated in FIG. 4 in that the sensing surface 15 is formed not by the metal film 40 but formed by the detection surface of the QCM sensor 132.

The QCM sensor 132 comprises a crystal oscillator 142 and an upper electrode 140 and a lower electrode 144 respectively formed on the upper and lower surfaces of the crystal oscillator 142. The upper electrode 140 is preferably a gold electrode. The surface of the upper electrode 140 is the detection surface of the QCM sensor 132, and QCM sensor 132 is attached to the inspection chip 16c in such a manner that the detection surface forms a part of the bottom surface of the channel of the inspection chip 16c.

The QCM sensor 132 is a mass sensor that measures the variation in mass attributable to the mass of a substance attached to the detection surface of the upper electrode 140 as a variation in frequency of the crystal oscillator 142; the QCM sensor 132 detects the variation in mass produced when the antigen 84, which, in the illustrated example, is bound to the secondary antibody, binds to the primary antibody 80 secured to the sensing surface 15 as a variation in resonant frequency of the crystal oscillator 142.

The variation in resonant frequency of the crystal oscillator 142 thus detected by the QCM sensor 132 is transmitted through the oscillation circuit 134 to the QCM analyzer 138, which calculates the binding quantity of the antigen 84 bound to the primary antibody 80 according to the variation in resonant frequency and further calculates the quantity or the concentration of the antigen 84, the analyte in the sample.

While, in Embodiments 1 to 3, the secondary antibody 88 labeled by the fluorescent substance 86 is disposed in the secondary antibody deposit region 49, the invention is not limited this way; without providing the secondary antibody deposit region 49, a liquid sample dropped to the leading end portion 47 may be a liquid sample wherein the analyte is previously labeled by the fluorescent substance 86.

### Embodiment 4

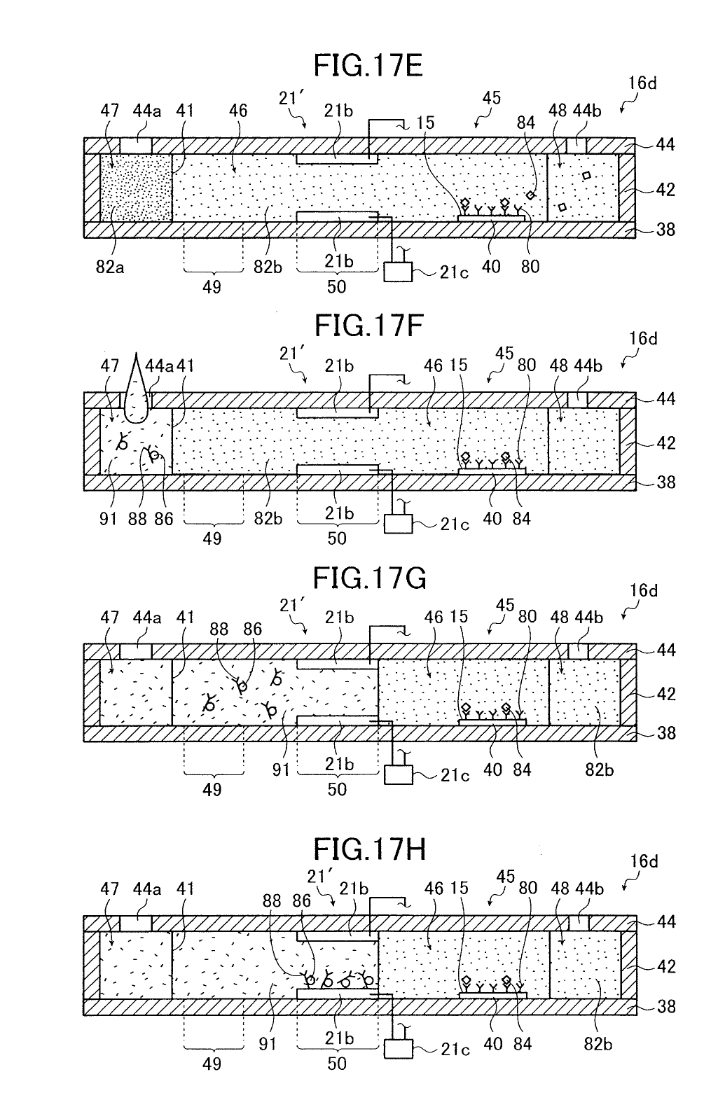

FIG. 16 is a view illustrating a configuration of an inspection kit 92 according to Embodiment 4. The inspection kit 92 comprises an inspection chip 16d and an ampoule 90 containing a labeling solution 91.

In the inspection chip 16, the liquid sample 82 is fed into the leading end portion 47 of the channel 45 as illustrated in FIG. 7A. In the inspection chip 16d, however, the liquid sample 82 is not fed, and the secondary antibody 88 labeled by the fluorescent substance 86 and the magnetic particles 87 is not disposed in the secondary antibody deposit region 49.

The ampoule 90 contains the labeling solution 91 including the secondary antibody 88 labeled by the fluorescent substance 86.

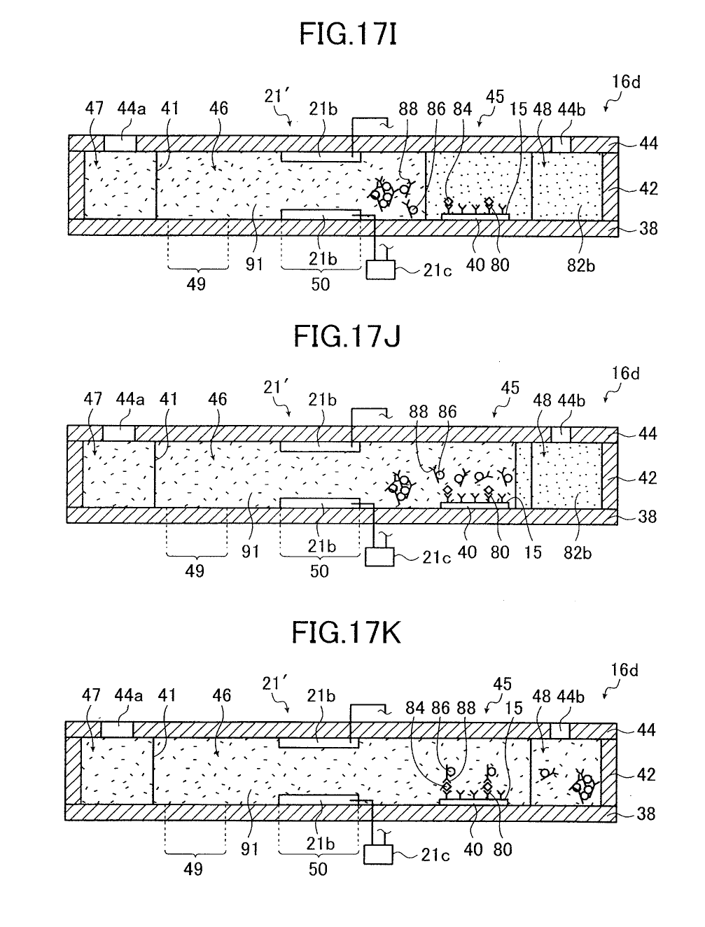

Next, a sensing method using an inspection kit 92 where the immunoassay is carried out using the sandwich technique will be described referring to FIGS. 17A to 17K.

A sensing device used comprises, for example, the light source 12, the incidence optics 14, the light detection unit 18, the computation unit 20, the FG 24 and the light source driver 26 illustrated in FIG. 1, the suction unit 22 illustrated in FIG. 5, and the concentration unit 21′ illustrated in FIG. 10A.

The fluorescent detection method, which is comparable to that used in Embodiment 1, will not be described here.

While the following is a case given as a representative example, where the liquid sample fed into the inspection chip 16d is blood 82a, the liquid sample may be any specimen, such as urine, that can be used for immunoassay using antigen-antibody reaction.

First, as illustrated in FIG. 17A, the blood (whole blood) 82a containing the analyte 84 is dropped from the feed inlet 44a of the cover 44 of the inspection chip 16d into the leading end portion 47 of the channel 45 of the channel substrate 42.

The blood 82a dropped into the leading end portion 47 is filtered by the blood cell filter 41, which passes plasma 82b and filters off red blood cells, white blood cells, etc. The plasma 82b then moves through the tube formed by the linear portion 46 and the transparent glass cover 44 because of the capillary shape thereof toward the terminal end portion 48.

Next, the suction unit 22 is attached to the terminal end portion 48 as illustrated in FIG. 5 to suck the plasma 82b in the channel 45 from the terminal end portion 48. The suction unit 22 is not shown in FIGS. 17A to 17K.

The plasma 82b moving from the leading end portion 47 of the channel 45 through the linear portion 46 of the channel 45 toward the terminal end portion 48 reaches the concentration region 50 of the linear portion 46 as illustrated in FIG. 17B.

When the liquid sample 82 reaches the concentration region 50, a voltage is applied from an electric power supply 21c to the electrodes 21b, 21b of the concentration unit 21′ to produce electric field as illustrated in FIG. 17C. In this state, the suction unit 22 is activated to perform suction so that the plasma 82b is caused to flow downwards, whereupon Coulomb's force is generated according to the electric charge of the analyte 84 and acts to move the analyte 84 toward the bottom surface of the inspection chip 16d, achieving concentration of the plasma 82b.

When a given quantity of the plasma 82b has flowed downwards, application of the electric voltage to the electrodes 21b, 21b of the concentration unit 21′ is stopped to terminate the concentration process.

This concentration process permits reduction of time for a reaction that is to follow in which the analyte 84 is caused to react with the primary antibody 80 located on the sensing surface 15 of the metal film 40.

Upon completion of the concentration, suction by the suction unit 22 causes the plasma 82b to flow further on through the linear portion 46 toward the terminal end portion 48 to reach the sensing surface 15 as illustrated in FIG. 17D.

The analyte 84 contained in the plasma 82b binds to the primary antibody 80 on the sensing surface 15.

As the suction unit 22 performs suction, the plasma 82b, after passing through the sensing surface 15 of the metal film 40, moves further on to the terminal end portion 48 as illustrated in FIG. 17E. The analyte 84 that is not captured by the primary antibody 80 also moves to the terminal end portion 48 along with the plasma 82b.

Next, as illustrated in FIG. 17F, the labeling solution 91 contained in the ampoule 90 and containing the secondary antibody 88 labeled by the fluorescent substance 86 is fed from the feed inlet 44a of the cover 44 of the inspection chip 16d to the leading end portion 47 of the channel 45 of the channel substrate 42.

The labeling solution 91, sucked by the suction unit 22, reaches the concentration region 50 of the linear portion 46 as illustrated in FIG. 17G.

When the labeling solution 91 reaches the concentration region 50, a voltage is applied from the electric power supply 21c to the electrodes 21b, 21b of the concentration unit 21′ to produce electric field as illustrated in FIG. 17H. In this state, the suction unit 22 is activated to perform suction so that the labeling solution 91 is caused to flow downwards, whereupon Coulomb's force, which is generated according to the electric charge of the secondary antibody 88 labeled by the fluorescent substance 86, moves the secondary antibody 88 labeled by the fluorescent substance 86 toward the bottom surface of the inspection chip 16d, achieving concentration of the labeling solution 91.

When a given quantity of the labeling solution 91 has flowed downwards, application of the electric voltage to the electrodes 21b, 21b of the concentration unit 21′ is stopped to terminate the concentration process.

This concentration process permits reduction of time for a reaction that is to follow in which the analyte 84 bound to the primary antibody 80 located on the sensing surface 15 of the metal film 40 is caused to react with the secondary antibody 88 labeled by the fluorescent substance 86.

The concentration may cause pieces of the secondary antibody 88 to gather together to form aggregates.

Upon completion of the concentration, suction by the suction unit 22 causes the labeling solution 91 to flow further on through the linear portion toward the terminal end portion 48 as illustrated in FIG. 17I.

As illustrated in FIG. 17J, the secondary antibody 88 labeled by the fluorescent substance 86 and the aggregates of the secondary antibody 88 are caused to flow toward the sensing surface 15 in this order, i.e., in increasing order of weight.

Taking advantage of the fact that the speed with which to arrive at the sensing surface 15 of the metal film 40 varies with the mass, the aggregates can be prevented from binding to the sensing surface 15 by stopping the flow of the labeling solution 91 or by completing the measurement before the aggregates reach the sensing surface 15 as in Embodiment 1 using the sandwich technique.

Upon arrival at the sensing surface 15, the secondary antibody 88 labeled by the fluorescent substance 86 binds to the analyte 84 bound to the primary antibody 80, as illustrated in FIG. 17K.

As the suction unit 22 performs suction, the labeling solution 91, after passing through the sensing surface 15 of the metal film 40, moves further on to the terminal end portion 48.

The aggregates of the secondary antibody 88 that are not captured by the analyte 84 which is bound to the primary antibody 80 and the aggregates of the secondary antibody 88 also move to the terminal end portion 48 along with the labeling solution 91.

In the process of concentrating the labeling solution 91, even when no aggregate of the secondary antibody 88 is formed, a so-called non-specific adsorption may occur whereby the secondary antibody 88 labeled by the fluorescent substance 86 binds directly to the metal film 40, possibly making accurate quantitative measurement of the analyte 84 impossible. However, a planned reaction can be accomplished in a short period of time by causing the secondary antibody 88 labeled by the fluorescent substance 86 to flow quickly across the metal film 40 by means of the suction performed by the pump 22.

Thus, the concentration of the analyte 84 can be detected by detecting the fluorescence in the same manner as in Embodiment 1 after binding the analyte 84 bound to the primary antibody 80 located on the sensing surface 15 and the secondary antibody 88 labeled by the fluorescent substance 86.

While, in the illustrated example, an electric field is generated by the concentration unit 21′ having the electrodes 21b, 21b to achieve concentration of the secondary antibody 88 labeled by the fluorescent substance 86, the secondary antibody 88 may be previously labeled by such a labeling substance for concentration that increases Coulomb's force acting upon the secondary antibody 88 by means of electric field. Further, the fluorescent substance 86 labeling the secondary antibody 88 may be previously labeled by such a labeling substance for concentration that increases Coulomb's force acting upon the fluorescent substance 86 by means of electric field.

Alternatively, the fluorescent substance 86 may be formed of fluorescent magnetic beads and the secondary antibody 88 may be labeled by the fluorescent magnetic beads to generate magnetic field using the concentration unit 21 that comprises the magnetic coil 12a as illustrated in FIG. 4 in order to achieve concentration of the secondary antibody 88.

While the inspection kit 92 is used in the above sensing method that employs the sandwich technique, the inspection kit 92 may be used in the competition technique.

### Other Embodiments

In Embodiments 1 to 4, the inspection chip is formed with a channel through which a sample liquid is caused to move from the leading end portion via the linear portion to the terminal end portion in order to bring the sample liquid into contact with the metal film. However, the invention is not limited this way and permits variations in configuration of the inspection chip.

The primary antibody may be secured directly to the surface of the prism instead of providing the metal layer (metal film) on the prism (dielectric). In this configuration, the liquid sample is brought into direct contact with the surface of the prism to which the primary antibody is secured, and the excitation light is allowed to enter the prism so as to be totally reflected at the interface between the prism and the liquid sample, thereby producing evanescent waves at the interface.

In Embodiments 1 to 4, the number of pieces of the analyte contained in the liquid sample or the concentration thereof is detected but the present invention is not limited thereto; presence or absence of the analyte in the sample may also be determined.

While, in Embodiment 1, the fluorescence produced by the fluorescent substance 86 excited by surface plasmons is detected, with the analyte bound to the secondary antibody labeled by the fluorescent substance, in order to detect the analyte, the method of labeling the analyte with the fluorescent substance is not limited specifically; for example, where the analyte itself is a fluorescent substance, the secondary antibody need not be provided.

Further, the invention may be applied to a sensing device of a type that detects scattered light (Raman scattered light) generated when surface plasmons are generated with the analyte attached to the metal film (or disposed close to the metal film).

In each of the foregoing embodiments, evanescent waves and/or surface plasmons are generated on the surface of the metal film and, furthermore, surface plasmon resonances are generated in order to form enhanced electric fields; however, the present invention is not limited thereto and may be applied to various approaches in which the intensity of enhancement varies with the angle of incidence of light at the surface where the enhanced electric fields are to be formed. For example, the present invention is applicable to a method whereby a gold film and a SiO₂ film about 1 μm thick are superposed on the prism and light admitted at a given angle is resonated in the SiO₂ film to form an enhanced electric field.

While, in Embodiment 1, the incidence optics are adjusted to admit the excitation light in such a manner that it is totally reflected by the metal film in order to optimally produce enhanced fields by means of surface plasmons, the invention is not limited this way; the excitation light may be admitted at an angle not causing total reflection.

What is claimed is:

1. A sensing method comprising the steps of:
allowing a liquid sample containing an analyte to flow through a channel,
applying a force oriented in a given direction normal to a direction in which the liquid sample flows in the channel upon the analyte in a given position of the channel to move the analyte in the given direction so that the analyte is concentrated,
causing the liquid sample to flow to a sensing surface forming a part of a wall surface of the channel located downstream of the given position and in the given direction against the channel, the sensing surface securing thereon a binding substance specifically reacting with the analyte, to allow the concentrated analyte to bind to the binding substance, and
detecting a quantity of the analyte bound to the binding substance.

2. The sensing method of claim 1, wherein the analyte is labeled by a labeling substance for concentration having one of an electric charge and a magnetism,
the step of concentrating the analyte including generating one of an electric field and a magnetic field in the given position of the channel and thus causing one of Coulomb's force and a magnetism to act upon the analyte to move the analyte in the given direction.

3. The sensing method of claim 2, wherein the labeling substance is magnetic particles.

4. The sensing method of claim 1, wherein in the detecting step, the quantity of the analyte is detected according to a detection light obtained upon emission of light toward the sensing surface.

5. The sensing method of claim 4, wherein the detection light is one of surface plasmon-induced scattered light and Raman scattered light both obtained, upon emission of the light, from the analyte bound to the binding substance, fluorescence generated from the analyte, and radiation light generated when surface plasmons are newly excited on the sensing surface by fluorescence produced from the analyte.

6. The sensing method of claim 4, wherein the analyte is labeled by a labeling substance for detection,
the detection light being one of fluorescence generated from the labeling substance for detection and radiation light generated when surface plasmons are newly excited on the sensing surface by fluorescence produced from the labeling substance for detection.

7. The sensing method of claim 4, wherein the light is emitted toward the sensing surface by one of an epi-illumination method, an evanescent illumination method, and a surface plasmon resonance illumination method.

8. The sensing method of claim 5, wherein the analyte is a substance capable of producing fluorescence.

9. The sensing method of claim 6, wherein the labeling substance for detection is a fluorescent labeling substance.

10. The sensing method of claim 6, wherein the labeling substance for detection is a scattering enhancement labeling substance.

11. The sensing method of claim 1, wherein the detection step comprises detecting a variation in resonant frequency of a crystal oscillator caused by binding the analyte to the binding substance secured to a surface of the crystal oscillator used as the sensing surface to detect the binding quantity of the analyte.

12. The sensing method of claim 1, wherein the flow of the liquid sample is stopped before aggregates formed in the concentration step reach the sensing surface.

13. The sensing method of claim 1, wherein the detection in the detection step is completed before aggregates formed in the concentration step reach the sensing surface.

14. A sensing device comprising:
a channel for allowing a liquid sample containing an analyte to flow therethrough,
concentration means for applying a force oriented in a given direction normal to a direction in which the liquid sample flows upon the analyte in a given position of the channel to move the analyte in the given direction so that the analyte is concentrated,
a sensing surface forming a part of a wall surface of the channel located downstream of the given position and in the given direction against the channel, the sensing surface securing thereon a binding substance specifically reacting with the analyte, and
detection means for detecting a quantity of the analyte bound to the binding substance.

15. The sensing device of claim 14, wherein the analyte is labeled by a labeling substance for concentration having one of an electric charge and a magnetism,
the concentrating means being one of electric field generating means for generating an electric field in the given position of the channel and magnetism generating means for generating a magnetic field in the given position of the channel.

16. The sensing device of claim 15, wherein the labeling substance is magnetic particles.

17. The sensing device of claim 14, wherein the detection means includes:
lighting means for irradiating the sensing surface with light;
a light detection unit for detecting a detection light obtained from the sensing surface; and
a computation unit for calculating a quantity of the analyte bound to the binding substance according to the detection light.

18. The sensing device of claim 17, wherein the optical detection unit detects as the detection light one of scattered surface plasmon-induced scattered light and Raman scattered light, upon emission of the light, from the analyte bound to the binding substance, fluorescence emitted from the analyte, and radiation light generated when surface plasmons are newly excited on the sensing surface by fluorescence produced from the analyte.

19. The sensing device of claim 17, wherein the analyte is labeled by a labeling substance for detection,
the light detection unit detecting as the detection light one of fluorescence generated from the labeling substance for detection and radiation light generated when surface plasmons are newly excited on the sensing surface by fluorescence produced from the labeling substance for detection.

20. The sensing device of claim 17, wherein the lighting means emits light toward the sensing surface by one of an epi-illumination method, an evanescent illumination method, and a surface plasmon resonance illumination method.

21. The sensing device of claim 19, wherein the labeling substance for detection is a fluorescent labeling substance.

22. The sensing device of claim 19, wherein the labeling substance for detection is a scattering enhancement labeling substance.

23. The sensing device of claim 14, wherein the detection means comprises a quartz crystal microbalance sensor including a crystal oscillator whose surface is used as the sensing surface.

24. The sensing device of claim 14, further comprising flow means for causing the liquid sample to flow through the channel.

25. The sensing device of claim 24, wherein the flow means stops the flow of the liquid sample before aggregates caused by the concentration means reach the sensing surface.

26. The sensing device of claim 24, wherein the flow means controls the flow of the liquid sample so that the detection by the detection means is completed before aggregates caused by the concentration means reach the sensing surface.

27. An inspection chip comprising:
a channel substrate including a channel through which a liquid sample containing an analyte is allowed to flow and formed with a feed inlet for feeding the liquid sample to the channel and a discharge outlet for discharging the liquid sample from the channel,
a sensing surface forming a part of a bottom surface of the channel between the feed inlet and the discharge outlet of the channel substrate,
a binding substance secured to the sensing surface and specifically reacting with the analyte, and
a concentration region located in the channel on a side of the sensing surface closer to the feed inlet and provided to apply a force to the analyte to move the analyte toward the bottom surface of the channel for concentration.

28. The inspection chip of claim 27 further comprising a labeling substance for concentration provided to label the analyte and placed upon the bottom surface of the channel between the concentration region and the feed inlet.

29. The inspection chip of claim 28, wherein the labeling substance is magnetic particles.

30. The inspection chip of claim 27 further comprising a first electrode disposed on the bottom surface of the channel in the concentration region and a second electrode disposed opposite the first electrode across the channel to generate an electric field in the concentration region.

31. An inspection kit comprising:
the inspection chip of claim 27 and
an ampoule containing a labeling solution including a labeling substance for detection for labeling the analyte for detection.

32. The inspection kit of claim 31, wherein the labeling substance for detection is labeled by the labeling substance for concentration so that the labeling substance for detection is concentrated in the concentration region of the inspection chip.

33. The inspection kit of claim 31, further comprising a first electrode disposed on the bottom surface of the channel in the concentration region of the inspection chip and a second electrode disposed opposite the first electrode across the channel to generate an electric field in the concentration region.
